SEQUENCE LISTING (2 sequences)

SEQ ID NO: 1 - protein, 121 aa, Artificial Sequence
  recombinant peptide
EVQLVESGGGLVQPGGSLRLSCAASGFTFSSSWMNWVRQAPGKGLEWVGRIYPGDGDTNYNVKFKGRFTISRDDSKNSLYLQMNSLKTEDTAVYYCARSGFITTVRDFDYWGQGTLVTVSS

SEQ ID NO: 2 - protein, 111 aa, Artificial Sequence
  recombinant polypeptide
EIVLTQSPDFQSVTPKEKVTITCRASESVDTFGISFMNWFQQKPDQSPKLLIHEASNQGSGVPSRFSGSGSGTDFTLTINSLEAEDAATYYCQQSKEVPFTFGGGTKVEIK

(19) United States
(12) Patent Application Publication
(10) Pub. No.: US 2022/0204617 A1
(43) Pub. Date: Jun. 30, 2022

(54) USE OF AN ANTI-CD19 ANTIBODY TO TREAT AUTOIMMUNE DISEASE

(71) Applicant: Viela Bio, Inc., Gaithersburg, MD (US)

(72) Inventors: Eliezer KATZ, Gaithersburg, MD (US); Jorn DRAPPA, Gaithersburg, MD (US); Kathleen MCKEEVER, Gaithersburg, MD (US)

(21) Appl. No.: 17/606,306

(22) PCT Filed: Apr. 23, 2020
(86) PCT No.: PCT/US20/29613

§ 371 Date: Oct. 25, 2021

Related U.S. Application Data
(60) Provisional application No. 62/838,093, filed on Apr. 24, 2019.
(60) Provisional application No. 62/843,096, filed on May 3, 2019.
(60) Provisional application No. 62/858,495, filed on Jun. 7, 2019.

(51) Int. Cl. C07K 16/28; A61P 25/00; A61P 27/02; A61K 39/395; A61K 45/06; A61K 49/00

(52) U.S. Cl. CPC C07K 16/2803; A61P 25/00; A61P 27/02; A61K 2039/505; A61K 45/06; A61K 49/0004; A61K 39/3955

(57) ABSTRACT

Methods for using an anti-CD19 antibody to treat autoimmune disease are disclosed herein. In particular the use of VIB551, a humanised, affinity-optimised, afucosylated IgG1 kappa monoclonal antibody to treat Neuromyelitis optica spectrum disorder.

### BACKGROUND

Neuromyelitis optica spectrum disorder (NMOSD) is a severe, autoimmune, inflammatory, central nervous system disease with a prevalence of 0.5-4.4/100 000.¹ NMOSD presents with optic neuritis, transverse myelitis,¹ and, less commonly, diencephalic, brainstem, and cerebral hemisphere attacks. Incomplete recovery from attacks is typical, and patients are at risk of death from respiratory failure.²

Once considered a multiple sclerosis variant, NMOSD is now recognised as a distinct disease,² characterised by astroglial injury, demyelination, and significant neuronal loss; most injury occurs during acute attacks.³ Highly specific serum autoantibodies against the astrocyte water channel aquaporin-4 (AQP4)-immunoglobulin G (IgG)⁴ are detected in 60-80% of patients and are likely pathogenic.⁵⁻⁷ In the presence of complement or inflammatory T-cell response, AQP4-IgG causes disease-specific central nervous system injury.⁵ Multiple lines of evidence suggest that NMOSD is predominantly a B-cell-mediated disorder resulting from pathologic autoantibody production, pro-inflammatory cytokine secretion, and B-cell antigen presentation.⁸

There are currently no approved therapies for NMOSD, and randomised, controlled trials have only been recently initiated. Immunosuppressants and B-cell depleting drugs, including rituximab,⁹ azathioprine,⁹ corticosteroids,⁹ and mycophenolate mofetil,⁹ are only empirically used to prevent attacks.

There is a clear unmet need for effective, evidence-based treatments to delay or prevent attacks in NMOSD.¹²

VIB551 is a humanised, affinity-optimised, afucosylated IgG1 kappa monoclonal antibody that binds to the B-cell surface antigen CD19. In contrast to anti-CD20 monoclonal antibodies that recognise and deplete a subset of CD20-expressing T lymphocytes (in addition to B lymphocytes),¹³ anti-CD19 antibodies recognise and deplete lymphocytes exclusively from the B-cell lineage.

It has now been discovered in an N-MOmentum clinical trial, that VIB551 is an effective B-cell depletion monotherapy for the prevention of relapses and disability in NMOSD. VIB551 has been shown to be superior over placebo in reducing: a) the risk of an NMOSD attack, b) the risk of disability worsening, c) MRI lesion activity and d) disease-related hospitalizations in a globally representative population of patients with NMOSD, recruited from 99 centres in 24 countries.

### BRIEF SUMMARY OF THE INVENTION

The description provides for a method of treating NMOSD. The method comprises a step of administering VIB551 to a patient in need of treatment for NMOSD, and a step of treating the NMOSD. The VIB551 is administered intravenously at a dose of 300 mg every 6 months or 26 weeks.

The description also provides for a method of reducing active magnetic resonance imaging (MRI) lesions in a patient diagnosed with NMOSD. The method comprises a step of administering VIB551 to a patient in need of treatment for NMOSD, and a step of reducing the MRI lesions in the patient. The VIB551 is administered intravenously at a dose of 300 mg every 6 months or 26 weeks.

The description further provides for a method of reducing AQP4-IgG titers in an AQP4-IgG⁺ patient in need of treatment NMOSD. The method comprises a step of administering VIB551 to a patient in need of treatment for NMOSD, and a step of reducing the AQP4-IgG titers in the patient. The VIB551 is administered intravenously at a dose of 300 mg every 6 months or 26 weeks.

The description provides for an additional method of treating patient diagnosed with NMOSD. The method comprises a step of administering VIB551 to a patient in need of treatment for NMOSD and a step of treating the NMOSD. The VIB551 is administered at a dose that (i) depletes at least 90% of circulating CD20+ B cells for at least six months, and (ii) does not increase risk of infections in the patient.

The description further provides for a method of reducing disability in a patient diagnosed with NMOSD. The method comprises a step of administering VIB551 to a patient in need of treatment for NMOSD and a step of reducing the disability in the patient. The VIB551 is administered intravenously at a dose of 300 mg every 6 months or 26 weeks.

The description further provides for a method of reducing NMOSD-related attacks in a patient in need of treatment for NMOSD. The method comprises a step of administering VIB551 to a patient in need of treatment for NMOSD and a step of reducing the NMOSD-related attacks in the patient. The VIB551 is administered intravenously at a dose of 300 mg every 6 months or 26 weeks

The description further provides for a method of monitoring NMOSD progression in a patient diagnosed with NMOSD. The method comprises a step of determining a first and a second number of MRI lesions in the patient. The NMOSD in the patient is identified as progressing if the second number of MRI lesions is greater than the first number of MRI lesions. The NMOSD in the patient is identified as non-progressing if the second number of MRI lesions is not greater than the first number of MRI lesions.

The description additionally provides a method of identifying a test agent as suitable for treating NMOSD in a patient diagnosed with NMOSD. A first number of MRI lesions is determined in the patient at most one month prior to treating with the test agent. A second number of MRI lesions is determined in the patient between three and twenty four months following the treating with the test agent. The test agent is identified as suitable for treating NMOSD if the second number of MRI lesions is the same as, or fewer than, the first number of MRI lesions. The test agent is identified as not suitable for treating NMOSD if the second number of MRI lesions is greater than the first number of MRI lesions.

### BRIEF DESCRIPTION OF FIGURES

FIG. 1. Flow chart of the multiplicity adjustment strategy in N-MOmentum clinical trial.

FIG. 2. Study design flow diagram for N-MOmentum clinical trial. (AC, adjudication committee; FU, follow-up; i.v., intravenous; max, maximum; min, minimum; NMO/NMOSD, neuromyelitis optica/neuromyelitis optica spectrum disorder; OLP, open-label period; RCP, randomised controlled period; Q26, every 26 (weeks); SFP, safety follow-up period)

FIG. 3. Flow diagram of attack assessment and adjudication in N-MOmentum (general). *MRI report/scans to be reviewed only when criteria require. †Rescue therapy can be initiated at any time at the discretion of the principal investigator. (AC, adjudication committee; AE, adverse event; EDSS, Expanded Disability Status Scale; FSS, functional systems score; MRI, magnetic resonance imaging; OLP, open-label period; RCP, randomised controlled period.)

FIG. 4. Flow diagram of attack assessment and adjudication related to NMOSD eye symptoms. (AE, adverse event; MRI, magnetic resonance imaging; NMO/NMOSD, neuromyelitis optica/neuromyelitis optica spectrum disorder; RAPD, relative afferent pupillary defect; RCP, randomised controlled period.)

FIG. 5. Flow diagram of NMO/NMOSD attack assessment and adjudication related to spinal cord symptoms. (AE, adverse event; EDSS, Expanded Disability Status Scale; FSS, functional systems score; MRI, magnetic resonance imaging; NMO/NMOSD, neuromyelitis optica/neuromyelitis optica spectrum disorder; RCP, randomised, controlled period).

FIG. 6. Flow diagram of NMO/NMOSD attack assessment and adjudication related to brain/brainstem symptoms. (AE, adverse event; EDSS, Expanded Disability Status Scale; FSS, functional systems score; MRI, magnetic resonance imaging; NMO/NMOSD, neuromyelitis optica/neuromyelitis optica spectrum disorder; RCP, randomised controlled period.

FIG. 7. N-MOmentum clinical trial CONSORT flow diagram. (*Efficacy endpoints were assessed in the intent-to-treat population, defined as participants who were randomised and received any investigational product and analysed according to their randomised treatment group, regardless of whether participants received an intervention other than the one planned. †Safety endpoints were assessed in the as-treated population, defined as participants who received any investigational product; however, participants randomised to VIB551 who received all placebo doses were included in the placebo group. Conversely, participants randomised to placebo who received at least one dose of VIB551 were included in the active treatment group. ‡Other includes one case each of need for treatment with prohibited medication, incorrect randomisation of an ineligible participant and withdrawal prior to dosing owing to occurrence of an attack on the day of randomisation (VIB551 arm), and patient decision (placebo arm). CONSORT, Consolidated Standards of Reporting Trials; i.v., intravenous; RCP, randomised controlled period.)

FIGS. 8A and 8B. Primary endpoint: time to adjudicated NMOSD attack. (A) Kaplan-Meier plot of the time to adjudicated NMOSD attack in the overall intent-to-treat population. (B) Kaplan-Meier plot of the time to adjudicated NMOSD attack in the AQP4-IgG seropositive population. Attacks were analysed using a Cox proportional hazards regression, with placebo as the reference group, and treatment and serotype as explanatory factors. (AQP4-IgG, aquaporin-4-immunoglobulin G; CI, confidence interval; NMOSD, neuromyelitis optica spectrum disorder).

FIGS. 9A and 9B. Effect of VIB551 and placebo on CD20+ B cells over 28 weeks in (A) the AQP4-IgG-seropositive population and (B) the overall intent-to-treat population. Data are shown as mean+standard deviation. Differences between treatment groups were significant from 4 weeks, p<0.0001. AQP4-IgG, aquaporin-4 immunoglobulin G; +ve, seropositive.

FIG. 10. VH (SEQ ID NO:1) and VL (SEQ ID NO:2) amino acid sequences of a VIB551 antibody.

FIGS. 11A and 11B. Median values for plasma cell-specific gene expression signature as fold-change from baseline over time, randomized controlled period (intent-to-treat population). FIG. 11A Median fold-change from baseline in inebilizumab and placebo-treated subjects plotted on a linear scale. FIG. 11B Median fold-change from baseline in inebilizumab and placebo-treated subjects plotted on a logarithmic scale. Error bars represent the 25% and 75% percentile of each treatment group. ITT=intent-to-treat.

FIG. 12. Median percent change from baseline in total immunoglobulin levels, randomized-controlled period (intent-to-treat population). Ig=immunoglobulin; Ineb=inebilizumab/VIB551; PBO=placebo.

FIG. 13. Median percent change from baseline in IgA levels, randomized-controlled period (intent-to-treat population). IgA=immunoglobulin A; Ineb=inebilizumab/VIB551; PBO=placebo.

FIG. 14. Median percent change from baseline in IgE levels, randomized-controlled period (intent-to-treat population). IgE=immunoglobulin E; Ineb=inebilizumab/VIB551; PBO=placebo.

FIG. 15. Median percent change from baseline in IgG levels, randomized-controlled period (intent-to-treat population). IgG=immunoglobulin G; Ineb=inebilizumab/VIB551; PBO=placebo.

FIG. 16. Median percent change from baseline in IgM levels, randomized-controlled period (intent-to-treat population). IgM=immunoglobulin M; Ineb=inebilizumab/VIB551; PBO=placebo.

FIG. 17. Median percent change from baseline in IgG levels, open-label period (open label population). IgG=immunoglobulin G; Ineb=inebilizumab/VIB551; PBO=placebo.

### DETAILED DESCRIPTION OF THE INVENTION

Described herein are VIB551 (also referred to MEDI551 or Inebilizumab) and its usefulness in methods for treating NMOSD, in methods for reducing active MRI lesions in a patient diagnosed with NMOSD, in methods of reducing AQP4-IgG titers in a AQP4-IgG⁺ patient in need of treatment for NMOSD, in methods of reducing NMOSD-related disability in a patient diagnosed with NMOSD, and in methods of reducing NMOSD-related attacks in a patient in need of treatment for NMOSD.

If VIB551 is used to treat NMOSD, it may treat the NMOSD by reducing the worsening of the patient's Kurtzke Expanded Disability Severity Scale (EDSS), or by reducing the number of active magnetic resonance imaging (MRI) lesions in the patient, or by reducing the worsening of the modified Rankin score of the patient, or by reducing the frequency of in-patient hospitalization of the patient related to NMOSD, or by reducing the risk of an NMOSD-related attack of the patient, or by reducing optic neuritis, or by reducing the severity of the patient's NMOSD-related attacks, or by decreasing the patient's pain, or by reducing NMOSD-related damage in the patient, or by reducing NMOSD-related attacks in the patient.

If the VIB551 treats the patient's NMOSD by reducing the worsening of the patient's EDSS score, and the patient has a baseline EDSS score of 0, then the patient's EDSS score may worsen by fewer than 2 points, or worsen by fewer than 1 point, or worsen by fewer than 0.5 points. This reduction of worsening of EDSS score for the patient with the baseline score of 0 may be over a time period of at least 6 months, 9 months, 1 year, 2 years, 3 years, 4 years, 5 years, 7.5 years, or 10 years. If the VIB551 treats the patient's NMOSD by reducing the worsening of the patient's EDSS score, and the patient has a baseline score of 1 to 5, then the patient's EDSS score may worsen by fewer than 1 point, or worsen by fewer than 0.5 point. This reduction of worsening for the patient with the baseline EDSS score of 1 to 5 may be reduction of worsening over a time period of over 6 months, 9 months, 1 year, 2 years, 3 years, 4 years, 5 years, 7.5 years, or 10 years. If the VIB551 treats the patient's NMOSD by reducing the worsening of the patient's EDSS score, and the patient has a baseline EDSS score of 5.5 or more, then the patient's EDSS score may worsen by fewer than 0.5 point or worsen by fewer than 0.25 point. This reduction of worsening for the patient with the baseline score of 5.5 or more may be reduction of worsening of EDSS score over a time period of over 6 months, 9 months, 1 year, 2 years, 3 years, 4 years, 5 years, 7.5 years, or 10 years.

If the VIB551 treats the patient's NMOSD by reducing the number of active MRI lesions, then the treating may be a reduction in the number of enlarging T2 MRI lesions or may be a reduction in the number of new MRI lesions, or may be a reduction in both the number of enlarging T2 MRI lesions and the number of new MRI lesions. The reduction in lesions may be a reduction in brain lesions, a reduction in brainstem lesions, a reduction in spinal cord lesions, a reduction in optic nerve lesions, or a reduction lesions in a combination of any two or more of brain, brainstem, spinal cord, and optic nerve. The new MRI lesions may not be clinically symptomatic.

If the VIB551 treats the patient's NMOSD by reducing the worsening of the patient's modified Rankin score, then the reducing of worsening may be such that the patient's modified Rankin score worsens by fewer than 2 points or by fewer than 1 point over a time period of at least 6 months, or of at least 9 months, or at least 1 year, or at least 2 years, or at least 3 years, or at least 4 years, or at least 5 years, or at least 7.5 years, or at least 10 years.

If the VIB551 treats the patient's NMOSD by reducing the risk of an NMOSD-related attack, then the patient's risk of attack may be reduced by between 60% and 85%, or may be reduced by between 65% and 75%, or may be reduced by between 70% and 80%. The patient's risk of attack may be reduced by at least 70%, at least 75%, at least 76%, at least 77%, at least 78%, at least 79% or at least 80%. The patient's risk of attack may be reduced by 70%, 75%, 76%, 77%, 78%, 79% or 80%.

If the VIB551 treats the patient's NMOSD by reducing the risk of an NMOSD-related attack, then as a result of the reduction in risk of an NMOSD-related attack, the treated patient's probability of having no NMOSD-related attack may be greater than 70% over at least 6 months following VIB551 treatment, or greater than 70% over at least 12 months following VIB551 treatment, or greater than 70% over at least 18 months following VIB551 treatment. As a result of the reduction in risk of an NMOSD-related attack, the treated patient's probability of having no NMOSD-related attack may be greater than 75% over at least 6 months following VIB551 treatment, or greater than 75% over at least 12 months following VIB551 treatment, or greater than 75% over at least 18 months following VIB551 treatment. In addition, as a result of the reduction in risk of an NMOSD-related attack, the treated patient's probability of having no NMOSD-related attack may be greater than 80% over at least 6 months following VIB551 treatment, or greater than 80% over at least 12 months following VIB551 treatment, or greater than 80% over at least 18 months following VIB551 treatment. Also, as a result of the reduction in risk of an NMOSD-related attack, the treated patient's probability of having no NMOSD-related attack may be greater than 85% over at least 6 months following VIB551 treatment, or greater than 85% over at least 12 months following VIB551 treatment, or greater than 85% over at least 18 months following VIB551 treatment.

Furthermore, if the VIB551 treats the patient's NMOSD by reducing the risk of an NMOSD-related attack, then as a result of the reduction in risk, the treated patient's annualized risk of an NMOSD-related attack may be reduced to between 0.18 and 0.08, or it may be reduced to between 0.15 and 0.08, or it may be reduced to 0.14, or 0.13, or 0.12, or 0.11, or 0.10, or 0.09, or 0.08, or 0.07. If the patient in treatment for NMOSD is AQP4-IgG seropositive, then the patient's annualized risk of an NMOSD-related attack may be reduced to between 0.15 and 0.11, or reduced to between 0.14 and 0.12, or it may be reduced to 0.14, 0.13, 0.12, or 0.11. If the patient in treatment for NMOSD is AQP4-IgG seronegative, then the patient's annualized risk of an NMOSD-related attack may be reduced to between 0.09 and 0.07, or it may be reduced to 0.09, 0.08, or 0.07.

The NMOSD-related attack, the risk of which may be reduced as in the treating of the NMOSD patient, may be an attack characterized by the appearance of a new NMOSD symptom or the worsening of an existing NMOSD symptom. The new or existing symptom may be an eye symptom. If the new or existing symptom is an eye symptom it may be eye pain, a new optic nerve lesion, an enlarging optic nerve lesion, blurred vision, loss of vision, or a 5 or more character drop in low-contrast Landolt C Broken Rings Chart. The new or existing symptom may be a spinal cord symptom. If the new or existing symptom is a spinal cord symptom, it may be a deep or radicular pain, extremity paraesthesia, weakness, sphincter dysfunction, Lhermitte's sign, a new spinal cord lesion, or an enlarging spinal cord lesion. The new or existing symptom may be a brain or brain stem symptom. If the new or existing symptom is a brain or brain stem symptom, it may be nausea, double vision, oculomotor palsy, vertigo, intractable vomiting, intractable hiccups, dysarthria, dysphagia, weakness, encephalopathy, hypothalamic dysfunction, a new brain or brain stem lesion, or an enlarging brain or brain stem lesion. The new or worsening symptom may a combination of any two or more of the eye, spinal cord, or brain/brain stem symptoms. It may be a combination of any two, three, or four of these symptoms.

If the VIB551 treats the patient's NMOSD by reducing optic neuritis, then the patient may experience reduced eye pain, reduced vision loss, reduced visual field loss, reduced loss of color vision, or reduced flashing or flickering of lights with movement of the eye. The reduction in optic neuritis may result in improved vision, and/or relief from eye pain.

If the VIB551 treats the patient's NMOSD by reducing the severity of the patient's NMOSD-related attacks, then the severity of any NMOSD-related attack suffered by the patient may be graded mild or moderate as opposed to graded as severe. A mild attack may be an attack that is transient, may be an attack that requires only minimal treatment or therapeutic intervention, and/or is an attack that may not interfere with usual activities of daily living. A moderate attack may be an attack that may be alleviated with specific additional therapeutic interventions. Any moderate attack may be one that interferes with usual activities of daily living, and/or causes discomfort, but that poses no significant or permanent risk of harm to the patient. The reduction of severity of the patient's NMOSD-related attacks may be a reduction in the attacks suffered by the patient as being graded as major. Such a major attack may be an attack that requires intensive therapeutic intervention, interrupts usual activities of daily living, or that significantly affects the clinical status of the patient. Such a major attack may require in-patient hospitalization.

If the VIB551 treats the patient's NMOSD by decreasing the patient's pain, then the decrease may be determined by a decrease in pain in the patient's eyes, legs, arms, upper back, and/or lower back. The decrease in pain may be in any one, any two, any three, any four, or all five of these regions. The decrease in pain may be measured by the pain numeric rating scale (PRS). The decrease in pain may be monitored relative to a baseline PRS level over a scale of 1 to 10. The decrease in pain may be a decrease in pain of at least one on the scale, at least 2 on the scale, at least 3 on the scale, at least 4 on the scale, or at least 5 on the scale. The decrease in pain may be a decrease in pain of between 1-5 on the scale, or a decrease in pain of between 1-3 on the scale, or a decrease in pain of between 1-2 on the scale.

If VIB551 treats the patient's NMOSD by reducing NMOSD-related damage, then the NMOSD-related damage may be the occurrence of a clinically asymptomatic new MRI lesion in the patient. If the NMOSD-related damage is the occurrence of a clinically asymptomatic new MRI lesion, it may occur in a patient who has not experienced symptoms of an NMOSD-related attack or in a patient who has experienced symptoms of an NMOSD-related attack.

If the clinically asymptomatic new MRI lesion occurs in a patient who has not experienced symptoms of an NMOSD-related attack, VIB551 may reduce the occurrence, or likelihood of occurrence, of a new clinically asymptomatic MRI lesion in any one or more domains, e.g., brain/brain stem, optic nerve or spinal cord, of the patient. The reduced occurrence, or likelihood of occurrence, of the new clinically asymptomatic MRI lesion may be a prevention of occurrence of the new clinically asymptomatic MRI lesion. The patient who has not experienced symptoms of an NMOSD-related attack, and whose NMOSD-related damage is reduced by VIB551, may be one who has not experienced symptoms of an NMOSD-related attack for at least 3 months, at least 6 months, at least 9 months, at least 12 month, at least 15 months, at least 18 months, at least 21 months or at least 24 months. Administration of VIB551 to the patient who has not experienced symptoms of an NMOSD-related attack may reduce the occurrence, or likelihood of occurrence, of a new clinically asymptomatic MRI lesion for the duration of the patient's treatment with VIB551. The administration of VIB551 to the patient who has not experienced symptoms of an NMOSD-related attack may result in a reduced occurrence, or likelihood of occurrence, of a new MRI lesion beginning within 1 month, within 2 months, or within 3 months following administration of a first dose of VIB551 and may continue for at least 6 months, at least 12 months, at least 18 months, at least 24 months, at least 30 months, at least 36 months, at least 42 months, at least at least 48 months, at least 54 months or at least 60 months following administration of the first dose of VIB551.

If the clinically asymptomatic new MRI lesion occurs in a patient who has experienced symptoms of an NMOSD-related attack, then the VIB551 may reduce the occurrence, or likelihood of occurrence, of a new clinically asymptomatic MRI lesion in a domain other than the domain in which the patient has experienced symptoms of the NMOSD-related attack. For instance, if the patient experienced symptoms of an NMOSD-related attack in the spinal cord, VIB551 may reduce the occurrence, or likelihood of occurrence, of a new MRI lesion in the optic nerve or brain/brain stem or both. If the VIB551 reduces, or reduces likelihood of, occurrence of a new clinically asymptomatic MRI lesion in association with an NMOSD-related attack, it may completely reduce, i.e., prevent, the occurrence of the new clinically asymptomatic MRI lesion in the patient. Moreover, VIB551 may not only reduce, or reduce likelihood of, a new clinically asymptomatic MRI lesion in the patent experiencing symptoms of the NMOSD-related attack, it may also reduce, or reduce likelihood of, occurrence of a new MRI lesion in the domain in which the patient experiences the symptoms of the NMOSD-related attack.

VIB551 may also be used in a method of reducing active MRI lesions in a patient diagnosed with NMOSD. If the VIB551 is used in a method of reducing active MRI lesions in a patient, then the VIB551 may reduce the cumulative total of new and enlarging lesions in the patient. If the VIB551 is used in a method of reducing active MRI lesions in a patient diagnosed with NMOSD, then the VIB551 may reduce the cumulative total of new gadolinium [Gd]-enhancing lesions, new T2 lesions and enlarging T2 lesions in the patient. If the VIB551 is used in a method of reducing active MRI lesions in a patient diagnosed with NMOSD, then the VIB551 may reduce the number of new T2 lesions in the patient and the number of enlarging T2 lesions in the patient. If the VIB551 is used in a method of reducing active MRI lesions in a patient diagnosed with NMOSD, then the VIB551 may reduce the number of new T2 lesions in the patient or the number an enlarging T2 lesions in the patient. The active MRI lesions reduced in the patient may be lesions in the brain/brain stem, spinal cord, and optic nerve cumulatively, or may be lesions in one or two of the brain/brain stem, spinal cord, or optic nerve. The active MRI lesions reduced in the patient may be clinically symptomatic or clinically asymptomatic MRI lesions. If the MRI lesions are clinically asymptomatic, they may be new MRI lesions that occur in a patient who has not experienced symptoms of an NMOSD-related attack. If the MRI lesions are clinically asymptomatic, they may be new MRI lesions that occur in a patient in association with an NMOSD-related attack, but not in the same domain as the domain in which the patient experiences the symptoms of the NMOSD-related attack.

The VIB551 may also be used in a method of reducing AQP4-IgG titers in a AQP4-IgG⁺ patient in need of treatment for NMOSD. If the VIB551 is used in a method of reducing AQP4-IgG titers in a AQP4-IgG⁺ patient in need of treatment for NMOSD, then the VIB551 may reduce the AQP4-IgG titers by 75% to 100%, or by 75% to 90%, or by 75% to 85%, or by 80% to 100%, or by 85% to 100%, or by 90% to 95%, or by 75%, 80%, 85%, 90%, 95 or 100%. The VIB551 may reduce the AQP4-IgG titers for a sustained period of time of at least 2 months, at least 3 months, at least 4 months, at least 5 months, at least 6 months, at least 7 months, at least 8 months, at least 9 months, at least 10 months, at least 11 months or at least 12 months following administration of a VIB551 dose.

The VIB551 may also be used in a method of reducing NMOSD-related disability in a patient diagnosed with NMOSD. The reducing of the NMOSD-related disability in the patient may be a reduction in the worsening of NMOSD-related disability in the patient or it may be a lessening of NMOSD-related disability in the patient. The NMOSD-related disability may be neurological disability or a manifestation of a neurological disability. The NMOSD-related disability may be characterized by one or more of eye pain, a loss of color vision, an overall loss of vision, blurred vision, double vision, overall weakness or paralysis, weakness or paralysis in the arms or legs, radicular pain, uncontrollable hiccups, uncontrollable nausea or vomiting, loss of bladder or bowel control, paralysis, and/or fatigue.

The reducing the NMOSD-related disability may be determined using EDSS, or may be determined using the modified Rankin Scale (mRS), or may be determined using EDSS and mRS. The reducing the NMOSD-related disability may be detectable within 6 to 12 months, within 6 to 8 months, or within 6 to 7 months of administering a dose of VIB551 or administering a first dose of VIB551.

If the reducing the NMOSD-related disability is determined using EDSS, the reducing in NMOSD-related disability may be either a reduction in the worsening in the patient's EDSS score or a lowering of the patient's EDSS score.

If the reducing the NMOSD-related disability is a reduction in the worsening in the patient's EDSS score, and the patient has a baseline EDSS score of 0, then the reduction in worsening may be such that, if the patient's EDSS score worsens, it worsens to a score of 0.5, or to a score of no more than 1, or to a score of no more than 1.5, or to a score of nor more than 2 over a period of time. The period of time in which the patient with the baseline score of 0 worsens to a score of 0.5, to no more than 1, to no more than 1.5, or to no more than 2 may be a period of time of at least 6 months, 9 months, 1 year, 1.5 years, 2 years, 3 years, 4 years, 5 years, 7.5 years, or 10 years. If the reducing the NMOSD-related disability is a reduction in the worsening in the patient's EDSS score, and the patient has a baseline EDSS score of 1 to 5, then the reduction in worsening may be a worsening of the patient's EDSS score by 0.5 points or by no more than 1 point over a period of time. The period of time in which the patient with the baseline score of 1 to 5 worsens by 0.5 points, or by no more than 1 point, may be a period of time of at least 6 months, 9 months, 1 year, 1.5 years, 2 years, 3 years, 4 years, 5 years, 7.5 years, or 10 years. If the reducing the NMOSD-related disability is a reduction in the worsening in the patient's EDSS score, and the patient has a baseline EDSS score of 5.5 or more, then the reduction in worsening may be a worsening of the patient's EDSS score by no more than 0.5 points. The period of time in which the patient with the baseline score of 5.5 worsens by the no more than 0.5 points may be a period of time of at least 6 months, 9 months, 1 year, 1.5 years, 2 years, 3 years, 4 years, 5 years, 7.5 years, or 10 years. The patient's baseline EDSS score may be determined within approximately 1 month, 2 weeks, 1 week, 3 days, 2 days, or 1 day of administration of a first VIB551 dose. The patient's baseline EDSS score may be determined coincident with administration of a first VIB551 dose or it may be determined within 1 day, within 2 days, within 3 days, within 1 week, within 2 weeks, or within 1 month of administration of a first VIB551 dose.

If the reducing the NMOSD-related disability is a lowering of the patient's EDSS score, then the patient's EDSS score may decrease by at least 0.5 points, or at least 1 point, or at least 1.5 points, or at least 2 points. The lowering or decreasing of the patient's EDSS score by the at least 0.5, at least 1, at least 1.5, or at least 2 points may occur over a period of time of approximately 2 weeks, approximately 1 month, approximately 2 months, approximately 3 months, approximately 4 months, approximately 5 months, approximately 6 months, approximately 9 months, approximately 12 months, or approximately 18 months. The period of time over which the patient's EDSS score is lowered, or that the patient's EDSS score decreases, may begin within 1 month, 2 weeks, 1 week, 3 days, 2 days, or 1 day of administration of a first VIB551 dose; or it may be coincident with administration of a first VIB551 dose; or it may begin within 1 day, within 2 days, within 3 days, within 1 week, within 2 weeks, or within 1 month of administration of a first VIB551 dose. The period of time over which the patient's EDSS score is lowered or decreases may begin at the time of an NMOSD attack, or may begin within 1 day, 2 days, 3 days, 4 days, 5 days, 6 days, or 7 days from the time of an NMOSD attack.

If the reducing the NMOSD-related disability is determined using mRS, then the reducing in NMOSD-related disability may be either a reduction in the worsening in the patient's mRS score or a lowering of the patient's mRS score.

If the reducing the NMOSD-related disability is a reduction in the worsening in the patient's mRS score, the reduction in worsening in mRS score may be may be a worsening of the patient's baseline mRS score by no more than 0.5 points or by no more than 1 point, or by no more than 1.5 points, or by no more than 2 points over a period of time. The period of time in which the patient's baseline mRS score worsens by the no more than 0.5 points, no more than 1 point, no more than 1.5 point, or no more than 2 points may be a period of time of at least 6 months, 9 months, 1 year, 1.5 years, 2 years, 3 years, 4 years, 5 years, 7.5 years, or 10 years. The patient's baseline mRS score, from which the reduction in worsening is determined, may be the patient's mRS score at approximately 1 month, approximately 2 weeks, approximately 1 week, approximately 3 days, approximately 2 days, or approximately 1 day prior to administration of a first VIB551 dose. The patient's baseline mRS score may be the patient's mRS score at the time of administration of a first VIB551 dose or it may be the patient's mRS score within 1 day, within 2 days, within 3 days, within 1 week, within 2 weeks, or within 1 month of administration of a first VIB551 dose.

If the reducing the NMOSD-related disability is a lowering of the patient's mRS score, then the patient's mRS score may lower or decrease by at least 0.5 points, or at least 1 point, or at least 1.5, or at least 2 points. The lowering or decrease of the patient's mRS score by the at least 0.5, at least 1, at least 1.5, or at least 2 points may be a lowering or decrease that occurs over a period of time of approximately 2 weeks, approximately 1 month, approximately 2 months, approximately 3 months, approximately 4 months, approximately 5 months, approximately 6 months, approximately 9 months, approximately 12 months, or approximately 18 months. The period of time over which the patient's mRS score is lowered may begin within 1 month, 2 weeks, 1 week, 3 days, 2 days, or 1 day of administration of a first VIB551 dose; or it may be coincident with administration of a first VIB551 dose; or it may occur within 1 day, within 2 days, within 3 days, within 1 week, within 2 weeks, or within 1 month of administration of a first VIB551 dose. The period of time over which the patient's mRS score is lowered may begin at the time of an NMOSD attack, or may begin within 1 day, 2 days, 3 days, 4 days, 5 days, 6 days, or 7 days from the time of an NMOSD attack.

The VIB551 may also be used in a method of reducing NMOSD-related attacks in a patient in need of treatment for NMOSD. The reducing of the NMOSD-related attacks in the patient may be a reduction in number of NMOSD-related attacks suffered by the patient in a first time period relative to a second time period. The first time period may occur following administration of a first VIB551 dose and the second time period may occur preceding the administration of the first VIB551 dose. The first time period may begin immediately following administration of a first VIB551 dose and the second time period may end immediately preceding the administration of the first VIB551 dose. The first and the second time period may be of an equal length in time. For example, the first and the second time period may both be at least 6 months, or 6 months, or at least 12 months, or 12 months, or at least 18 months, or 18 months, or at least 24 months, or 24 months, or at least 30 months, or 30 months, or at least 36 months, or 36 months, or at least 42 months, or 42 months, or at least 48 months, or 48 months, or at least 54 months or 54 months, or at least 60 months or 60 months.

The reduction in number of NMOSD-related attacks suffered by the patient in the first time period relative to the second time period may such that the patient suffers at least 1 fewer attack, or 1 fewer attack, or at least 2 fewer attacks, or 2 fewer attacks, or at least 3 fewer attacks, or 3 fewer attacks, or at least 4 fewer attacks, or 4 fewer attacks, or at least 5 fewer attacks, or 5 fewer attacks in the first time period relative to the second time period. The NMOSD-related attacks suffered by the patient in the first and/or the second time period may be NMOSD-related attacks that are optic neuritis attacks, myelitis attacks, or brainstem attacks. If the patient suffers an NMOSD-related attack in the first time period, the NMOSD-related attack may or may not be in the same domain, e.g., optic nerve, spinal cord or brain/brain stem, as an NMOSD-related attack(s) suffered by the patient in the second time period.

The NMOSD-related attacks suffered by the patient, the number of which may be reduced in the patient in need of treatment for NMOSD, may be NMOSD-related attacks characterized by the appearance of a new NMOSD symptom, or the worsening of an existing NMOSD symptom, or the appearance of a new MRI lesion that may or may not be symptomatic.

If the NMOSD-related attack is an attack characterized by a new symptom or the worsening of an existing symptom, the new or worsening symptom may be an eye symptom. If the new or worsening symptom is an eye symptom it may be eye pain, a new optic nerve lesion, an enlarging optic nerve lesion, blurred vision, loss of vision, or a 5 or more character drop in low-contrast Landolt C Broken Rings Chart. The NMOSD-related attack, characterized by a new eye symptom or the worsening of an existing eye symptom, may further/alternatively meet any one or more of the following criteria: >15-character drop in high-contrast Landolt C Broken Ring Chart from most recent clinical visit as measured in a previously affected eye and no other ophthalmological explanation, Reduction of ≥2 steps‡ in CF to NLP from most recent clinical visit as measured in a previously affected eye and no other ophthalmological explanation, Reduction of ≥7 characters in low-contrast Landolt C Broken Ring Chart from most recent clinical visit as measured in either eye alone (monocular) AND a new RAPD in affected eye, Reduction of ≥7 characters in low-contrast Landolt C Broken Ring Chart from most recent clinical visit as measured in either eye alone (monocular) AND loss of a previously documented RAPD in fellow eye, Reduction of ≥5 characters in high-contrast Landolt C Broken Ring Chart from most recent clinical visit as measured in either eye alone (monocular) AND a new RAPD in affected eye, Reduction of ≥5 characters in high-contrast Landolt C Broken Ring Chart from most recent clinical visit as measured in either eye alone (monocular) AND loss of a previously documented RAPD in fellow eye, Reduction of ≥1 step§ in CF to NLP from most recent clinical visit as measured in a previously affected eye AND a new RAPD in affected eye, Reduction of ≥1 step§ in CF to NLP from most recent clinical visit as measured in a previously affected eye AND loss of a previously documented RAPD in fellow eye, Reduction of ≥7 characters in low-contrast Landolt C Broken Ring Chart from most recent clinical visit as measured in either eye alone (monocular) AND a new Gd-enhancing or new/enlarging T2 MRI lesion in the corresponding optic nerve, Reduction of ≥5 characters in high-contrast Landolt C Broken Ring Chart from most recent clinical visit as measured in either eye alone (monocular) AND a new Gd-enhancing or new/enlarging T2 MRI lesion in the corresponding optic nerve, Reduction of ≥1 step§ in CF to NLP from most recent clinical visit as measured in a previously affected eye AND a new Gd-enhancing or new/enlarging T2 Mill lesion in the corresponding optic nerve.

If NMOSD-related attack is an attack characterized by a new symptom or the worsening of an existing symptom, the new or worsening symptom may be a spinal cord symptom. If the new or worsening symptom is a spinal cord symptom, it may be a deep or radicular pain, extremity paraesthesia, weakness, sphincter dysfunction, Lhermitte's sign, a new spinal cord lesion, or an enlarging spinal cord lesion. The NMOSD-related attack, characterized by a new spinal cord symptom or the worsening of an existing spinal cord symptom, may further/alternatively meet any one or more of the following criteria: Worsening of ≥2 points in at least one of the relevant (pyramidal, bladder/bowel, sensory) FSS compared with most recent clinical visit, Worsening of ≥1 point in EDSS score compared with most recent clinical visit if previous EDSS score ≥5.5, Worsening of ≥1 point in at least two of the relevant (pyramidal, bladder/bowel, sensory) FSS compared with most recent clinical visit when the most recent clinical visit score was ≥1 AND a new Gd-enhancing or new/enlarging T2 MRI lesion in the spinal cord, Worsening of ≥0.5 points in EDSS score compared with most recent visit if previous EDSS score ≥5.5 AND a new GD-enhancing or new/enlarging T2 MRI lesion in the spinal cord.

If NMOSD-related attack is an attack characterized by a new symptom or the worsening of an existing symptom, the new or worsening symptom may be a brain or brain stem symptom. If the new or existing symptom is a brain or brain stem symptom, it may be nausea, double vision, oculomotor palsy, vertigo, intractable vomiting, intractable hiccups, dysarthria, dysphagia, weakness, encephalopathy, hypothalamic dysfunction, a new brain or brain stem lesion, or an enlarging brain or brain stem lesion. The NMOSD-related attack, characterized by a new brain/brain stem symptom or the worsening of an existing brain/brain stem symptom, may further/alternatively meet any one or more of the following criteria: Isolated (not present at most recent clinical visit) intractable nausea, vomiting, and/or hiccups lasting >48 hours AND a new Gd-enhancing or new/enlarging T2 MRI lesion in the brainstem, Worsening of ≥2 points in at least one of the relevant (brainstem, cerebellar) FSS compared with most recent clinical visit AND a new Gd-enhancing or new/enlarging T2 MRI lesion in the brainstem, or Worsening of ≥2 points in at least one of the relevant (cerebral, sensory, pyramidal) FSS (with a score of ≥3 at the current visit) compared with most recent clinical visit AND a new Gd-enhancing or new/enlarging T2 MRI lesion in the brain consistent with the clinical presentation.

The NMOSD-related attack may be an attack characterized by any combination of new and/or worsening symptoms of any one, two, or more of the eye, spinal cord, or brain/brain stem. The NMOSD-related attack may be an attack characterized by any combination of two, three, or four of the symptoms or other criteria identified for any of the one or more of the eye, spinal cord or brain/brain stem.

Furthermore, the NMOSD-related attack may be an attack characterized by the appearance of new MRI lesions in the patient. The new MRI lesions may, but are not necessarily, symptomatic.

The VIB551 administered in the methods of treating NMOSD, methods of reducing active MRI lesions in an NMOSD patient, methods of reducing AQP4-IgG titers in a AQP4-IgG⁺ NMOSD patient, methods of reducing disability in a patient diagnosed with NMOSD, and methods of reducing NMOSD-related attacks in a patient in need of treatment for NMOSD may be at an interval of approximately every 6 months and may be intravenous. The approximately every 6 months may be administration every 6 months, every 180 days, every between 170 and 190 days, every between 175 and 185 days, every between 175 and 190 days, or every between 170 and 185 days. The approximately every 6 months may be administration every 26 weeks, every 25 weeks, every 27 weeks, every between 25 and 27 weeks, every between 25 and 26 weeks, or every between 26 and 27 weeks. Prior to the administering the VIB551 every approximately 6 months in the method, an initial VIB551 dose may be administered to the NMOSD patient. The initial VIB551 dose may be administered approximately 2 weeks before the approximately every 6 month VIB551 dosing. The administering the initial VIB551 dose approximately 2 weeks before the every approximately 6 month VIB551 dosing may be the administering of the initial VIB551 dose 12 days, 13 days, 14 days, 15 days, or 16 days before the approximately 6 months VIB551 dosing. The initial VIB551 dose may or may not be co-administered with oral corticosteroids.

The dose VIB551 administered in the methods of treating NMOSD, methods of reducing active MRI lesions in an NMOSD patient, methods of reducing AQP4-IgG titers in a AQP4-IgG⁺ NMOSD patient, and methods of reducing disability in a patent diagnosed with NMOSD may be a dose of approximately 300 mg. The approximately 300 mg may be a dose of 250 mg to 350 mg, it may be a dose of 275 to 325 mg, it may be a dose of 290 to 310 mg, it may be a dose of 205 to 305 mg, or it may be a dose of 300 mg.

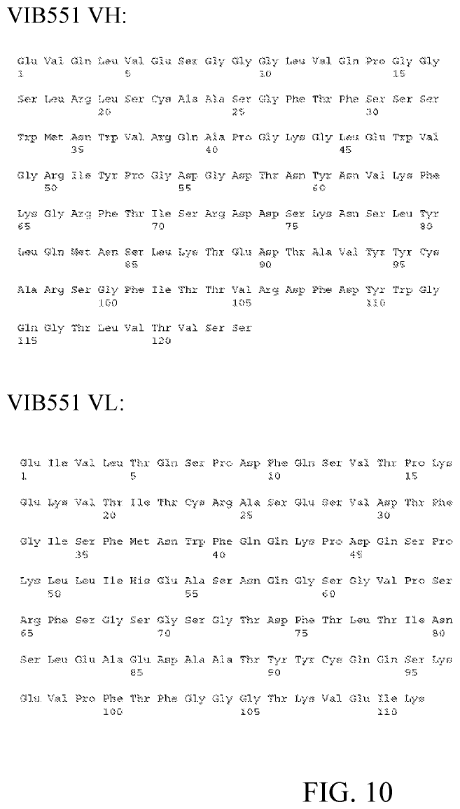

The VIB551 administered in the methods of treating NMOSD, methods of reducing active MRI lesions in an NMOSD patient, methods of reducing AQP4-IgG titers in a AQP4-IgG⁺ NMOSD patient, and methods of reducing disability in a patient diagnosed with NMOSD may have a VH amino acid sequence and a VL amino acid sequence as shown in FIG. 10. The VIB551 administered in the methods may have the VH amino acid sequence and the VL amino acid sequence as shown in FIG. 10, but for one or more changes in amino acid residues that do not alter the function of the VIB551 amino acid sequence. The number of amino acid changes may be 1 amino acid residue change, 2 amino acid residue changes, 3 amino acid residue changes, 4 amino acid residue changes, or 5 amino acid residue changes. The VIB551 administered in the methods may have the CDR amino acid sequences of the VH and the VL sequences as shown in FIG. 10, but may have one or more alterations in the framework regions of the VH and the VL sequences shown in FIG. 10.

The VIB551 administered in the methods of treating NMOSD, methods of reducing active MRI lesions in an NMOSD patient, methods of reducing AQP4-IgG titers in a AQP4-IgG⁺ NMOSD patient, and methods of reducing disability in a patient diagnosed with NMOSD may be packaged in a 10-mL vial filled with a nominal 10-mL solution of VIB551 at a concentration of 10 mg/mL, containing 20 mM histidine/histidine hydrochloride, 70 mMNaCl, 106 mM (4% [w/v]) trehalose dehydrate, and 0.01% (w/v) polysorbate 80, pH 6.0.

The NMOSD patient to which the VIB551 is administered in the methods of treating NMOSD, the methods of reducing active MRI lesions in an NMOSD patient and the methods of reducing NMOSD-related disability in a patient diagnosed with NMOSD may or may not be AQP4-IgG seropositive. The NMOSD patient may be screened for AQP4-IgG prior to VIB551 administration.

The VIB551 may also be used in a method of treating a patient in need of treatment for NMOSD in which the VIB551 is administered at a dose that (i) depletes at least 90% of circulating CD20+ B cells for at least six months, and (ii) does not increase risk of infections in the patient. The dose that depletes the at least 90% of circulating CD20+ B cells for at least six months may also deplete peripheral blood CD20⁻ plasmablasts and plasma cells. The dose that depletes the at least 90% of circulating CD20+ B cells for at least six months may also reduce the plasma cell gene signature of the patient in need of treatment for NMOSD, or it may ablate the plasma cell gene signature of the patient in need of treatment for NMOSD. The dose that depletes the at least 90% of circulating CD20+ B cells may deplete the circulating CD20+ B cells for longer than six months. It may deplete the at least 90% of circulating CD20+ B cells for at least 9 months or at least 1 year.

The dose of VIB551 that depletes at least 90% of circulating CD20+ B cells for at least six months in the method of treating also does not increase risk of infections in the NMOSD patient. The risk of infection may not be increased in the NMOSD patient relative to his or her risk of infections prior to the administration of VIB551. The risk of infection may not be increased in the NMOSD patient in comparison to an NMOSD patient not treated with VIB551. The risk of infection may be a risk of infection by or resulting in typical pneumonia, beta haemolytic streptococcal infection, bronchitis, conjunctivitis, viral conjunctivitis, fungal skin infection, gastroenteritis viral, gastrointestinal infection, gingivitis, cystitis, herpes zoster, influenza, laryngitis, meningitis viral, muscle abscess, oral herpes, otitis externa, periodontitis, pneumonia, rhinitis, retinitis, pyelocystitis, retinitis, sinusitis, urinary tract infection, tinea cururis, septic shock, or upper respiratory tract infection.

The dose of VIB551 that may be used in a method of treating a patient in need of treatment for NMOSD, wherein the VIB551 dose depletes at least 90% of circulating CD20+ B cells for at least six months, and does not increase risk of infections in the patient, may be a dose of approximately 300 mg. The approximately 300 mg may be a dose of 250 mg to 350 mg, it may be a dose of 275 to 325 mg, it may be a dose of 290 to 310 mg, it may be a dose of 205 to 305 mg, or it may be a dose of 300 mg.

The dose of VIB551 that may be used in a method of treating a patient in need of treatment for NMOSD, wherein the VIB551 dose depletes at least 90% of circulating CD20+ B cells for at least six months, and does not increase risk of infections in the patient, may be a dose administered intravenously at an interval of approximately every 6 months, or every 7 months, or every 8 months, or every 9 months, or every 10 months, or every 11 months, or once a year.

In addition to the present description providing VIB551 and its usefulness in methods relating to treatment of a patient diagnosed with NMOSD, e.g., by treating NMOSD or reducing active MRI lesions or reducing AP4-IgG titers or reducing NMOSD-related disability of the patient, the present description also provides methods of monitoring NMOSD progression and methods of identifying a test agent as suitable for treating NMOSD in a patient.

In a method of monitoring NMOSD progression, NMOSD is monitored in a patient diagnosed with NMOSD. The monitoring of NMOSD progression may be performed by determining a first and a second number of MRI lesions in the patient. The first and second number of MRI lesions in the patient may be determined at time interval apart of between 6 and 24 months, or between 6 and 18 months, or between 6 and 12 months, or between 12 and 24 months, or between 18 and 24 months. The first and the second number of MRI lesions in the patient may be determined at a time interval apart of approximately 6 months, approximately 9 months, approximately 12 months, approximately 15 months, approximately 18 months, approximately 24 months, approximately 30 months or approximately 36 months. The first and second MRI lesions may include clinically asymptomatic MRI lesions.

The first number of MRI lesions may or may not be determined prior to a first dose of a treatment. If the first number of MRI lesions is determined prior to a first dose of a treatment, the first number of MRI lesions may be determined approximately 1 month, 3 weeks, 2 weeks, 1 week, 6 days, 5 days, 4 days, 3 days, 2 days, or a day prior to the first dose of the treatment. The second number of MRI lesions may then be determined between 6 and 24 months, or between 6 and 18 months, or between 6 and 12 months, or between 12 and 24 months, or between 18 and 24 months following the first dose of the treatment. The second number of MRI lesions may be determined 6 months, 9 months, 12 months, 15 months, 18 months, 24 months, 30 months or 36 months following the first dose of the treatment.

Having determined the first and the second number of MRI lesions, the patient may be identified as progressing if the second number of MRI lesions is greater than the first number of MRI lesions. Alternatively, the patient may be identified as not progressing if the second number of MRI lesions is not greater than the first number of MRI lesions.

If the first number of MRI lesions was determined prior to a first dose of a treatment, and the second number of MRI lesions is greater than the first number of MRI lesions, then the patient may further be identified as a non-responder to the treatment. Alternatively, if the first number of MRI lesions was determined prior to a first dose of a treatment, and the second number of MRI lesions is not greater than the first number of MRI lesions, then the patient may be identified as a responder to the treatment.

The patient, whose NMOSD progression is monitored, may or may not experience clinical symptoms of an NMOSD attack, i.e., may be clinically asymptomatic, throughout the time interval between determination of the first and the second number of MRI lesions. If the patient is clinically asymptomatic throughout the time interval, and the second number of MRI lesions is greater than the first number of MRI lesions, the patient may be identified as progressing in the absence of clinical symptoms of an attack. Furthermore, even if the patient is clinically asymptomatic, if the second number of MRI lesions is greater than the first number of MRI lesions in the patient, the patient may be identified as having had an NMOSD attack. For example, if the patient is determined to have 1 or more, 2 or more, 3 or more, 4 or more, or determined to have 5 or more second MRI lesions than first MRI lesions, the patient may be identified as progressing and/or as having had at least one NMOSD attack.

If the patient is identified as progressing and/or identified as having had at least one NMOSD attack, i.e., having more second MRI lesions than first MRI lesions, the patient may be treated. If the patient is treated, a therapeutic agent may be administered to the patient. The therapeutic agent may be, for example, steroids, eculizumab, satralizumab, or VIB551. If the therapeutic agent is VIB551, the VIB551 may be administered to the patient intravenously at a dose of 300 mg every 6 months or administered according to any other VIB551 dose/dose schedule described herein. If the patient is identified as progressing and/or identified as having had at least one NMOSD attack and the patient is further identified as a nonresponder to a treatment, the patient may discontinue the treatment and may be administered an initial dose of a second treatment or may be administered an initial dose of the second treatment in combination with the first treatment.

In a method of identifying a test agent as suitable for treating NMOSD in a patient diagnosed with NMOSD, a first number and a second number of MRI lesions is determined in the patient. The first number of MRI lesions is determined prior to the treating of the patient with the test agent. The first number of MRI lesions may be determined at most 1 month prior to the treating the patient with the test agent. The first number of MRI lesions may be determined at most 3 weeks, at most 2 weeks, at most 1 week, at most 6 days, at most 5 days, at most 4 days, at most 3 days, at most 2 days or at most 1 day prior to the treating the patient with the test agent. Following the treating with the test agent, the second number of MRI lesions is determined. The second number of MRI lesions in the patient may be determined between 3 months and 24 months following the treating with the test agent. The second number of MRI lesions in the patient may be determined between 6 months and 24 months, or between 9 months and 24 months, or between 12 months and 24 months, or between 15 months and 24 months, or between 18 months and 24 months, or between 21 months and 24 months, or between 3 months and 21 months, or between 3 months and 18 months, or between 3 months and 15 months, or between 3 months and 12 months, or between 3 months and 9 months, or between 3 months and 6 months following the treating with the test agent. The second number of MRI lesions in the patient may be determined 3 months, 6 months, 9 months, 12 months, 15 months, 18 months, 21 months, or 24 months following the treating with the test agent.

The test agent may be identified as suitable for treating NMOSD if the second number of MRI lesions is the same or fewer than the first number of MRI lesions. The test agent may not be identified as suitable for treating NMOSD if the second number of MRI lesions is greater than the first number of MRI lesions. The first and/or second MRI lesions may include clinically asymptomatic MRI lesions.

Those skilled in the art will recognize, or be able to ascertain using no more than routine experimentation, many equivalents to the specific embodiments described herein. Such equivalents are intended to be encompassed by the appended claims.

All publications, patents and patent applications mentioned in this specification are herein incorporated by reference into the specification to the same extent as if each individual publication, patent or patent application was specifically and individually indicated to be incorporated herein by reference.

### EXAMPLES

### Example 1—Rationale for Elements of Clinical Trial Design

Overview. An NMOSD clinical trial, N-MOmentum, was designed as a randomised, placebo-controlled, double-blind, 197-day, phase 2/3 study with an open-label extension period to assess the efficacy and safety of VIB551 (also referred to as VIB551 or MedI551), an anti-CD19, B-cell depleting antibody, in patients with NMOSD, recruited from 99 sites in 24 countries. Participants were randomised (3:1) using an interactive voice response system/interactive web response system, to intravenous VIB551 300 mg or placebo, respectively, administered on Days 1 and 15. Efficacy endpoints were assessed in the intent-to-treat population, and safety endpoints in the as-treated population. The primary endpoint was time to first adjudicated attack; secondary endpoints included disability worsening, magnetic resonance imaging (MRI) lesion activity, and hospitalisations.

Arm selection. The placebo-comparator treatment arm was chosen because there are no currently approved medications for the treatment of neuromyelitis optica spectrum disorder. The use of a placebo arm allowed for a clear and robust evaluation of VIB551, avoiding the confounding effects of other treatments, providing the highest sensitivity and robustness to detect efficacy, and helping to deliver a clinically meaningful outcome of this study.

Randomization. The 3:1 randomisation ratio used in this study was an effective and efficient approach to build an enriched safety database for VIB551 while keeping the number of required events or patients in the placebo arm at a minimum acceptable level. This randomisation ratio also addressed, to a certain degree, the ethical concerns of investigators and patients regarding enrollment of patients to a placebo arm. In addition to limiting the number of patients who received placebo, the study was designed to limit the actual duration of placebo exposure to a maximum of 197 days or time to onset of an attack, whichever occurred earlier, after which all patients had the option to enter the open-label period and to receive VIB551.

Prior to randomisation, patients were stratified based on AQP4-IgG serostatus (determined at screening) and region (Japan vs non-Japan). Within each stratum, patients were randomised in a 3:1 ratio using an interactive voice response system/interactive web response system (IVRS/IWRS) with a permuted block randomisation scheme to a treatment group and assignment of blinded investigational product kit numbers. A patient was considered randomised into the study when the investigator notified the IVRS/IWRS that the patient met eligibility criteria and the IVRS/IWRS provided the assignment of masked investigational product kit numbers to the patient.

Blinding. This was a double-blind study. VIB551 and placebo were identically labelled and indistinguishable in appearance; both were supplied as clear to opalescent, colourless to yellow liquids, and free from or practically free from particles. VIB551 and placebo doses were indistinguishable during dose preparation, handling, and infusion.

Neither the patient/legal representative nor any of the investigator or sponsor staff who were involved in the treatment or clinical evaluation of the patients were aware of the treatment received. In the event that treatment allocation for a patient became known, the sponsor was notified immediately.

Administration of the blinded dose of VIB551 or placebo on open-label period day 15 was necessary to correctly administer the loading dose of VIB551 600 mg i.v. to patients previously randomised to placebo, or to ensure that patients previously randomised to VIB551 did not receive an extra treatment dose. This blinding mechanism was implemented through the IVRS to ensure that details of the randomised treatment were not revealed to the sites.

VIB551 is known to deplete CD19+ B cells; therefore, the results of flow cytometry to count B cells were potentially unblinding. These data were not made available to investigation sites post randomisation throughout the remainder of the study.

Data from early phase development in non-oncology patient populations receiving VIB551 suggested that administration could be associated with a potential unspecified mild reduction in total immunoglobulin in individual patients. These data were not made available to investigational sites post randomisation throughout the remainder of the study because the reduction could have been potentially unblinding.

VIB551 is hypothesised to reduce titres of AQP4-IgG. AQP4-IgG titres from the central laboratory were not made available to investigational sites at any point in the study.

VIB551 is known to reduce the plasma cell gene signature. Plasma cell gene signature assay samples were not tested before the unblinding of the study because the results could have been potentially unblinding.

VIB551 may also reduce tetanus vaccine titres; therefore, the results of the vaccine titre assay were not made available to the sites at any point during the study.

Any potentially unmasking data from the study were not made available to the sponsor until the database was locked and the study had been unblinded following completion of the randomised controlled period.

An unblinded interim analysis was included in the study design. The interim analysis to determine futility was conducted by the data monitoring committee; the sponsor and sites remained blinded to the treatment assignments post interim analysis.

Built-in safety mechanisms. Owing to the potential severity of neuromyelitis optica spectrum disorder attacks and their debilitating nature, the study was further designed to ensure safety in this patient population through: careful monitoring and early evaluation of signs and symptoms of neuromyelitis optica spectrum disorder attacks; scheduled study visits and study assessments; follow-up telephone contact every 2 weeks with study patients by study site staff; an ‘escape clause’ (i.e. immediate access to rescue therapy following identification of a neuromyelitis optica spectrum disorder attack); and monitoring by an independent data monitoring committee.

Comparator. Placebo was the chosen comparator following comprehensive ethical consideration; there were no approved therapies or controlled trials performed to establish the risk/benefit profile of off-label medications.^{1,18}

Discontinuation. Treatment was discontinued if participants withdrew consent, or if the investigator determined an adverse event that precluded further dosing, including elevated liver aminotransferase levels, severe anaphylaxis, hypersensitivity reaction, infusion reaction, neutropenia, and pregnancy. Specifically, a subject did not receive further investigational product if any of the following occurred in the patient in question: Withdrawal of consent for further treatment with investigational product or the patient was lost to follow-up; An adverse event that, in the opinion of the investigator or the sponsor, contraindicated further dosing; The patient was determined to have met one or more of the exclusion criteria or failed to meet all the inclusion criteria for study participation and there was a potential safety risk associated with continuation identified upon consultation with the medical monitor; Any of the following liver function abnormalities: (a) ALT or AST >8×ULN, (b) ALT or AST >5×ULN for >2 weeks (in absence of elevated bilirubin and/or other symptoms listed in item ‘d’), (c) ALT or AST >3×ULN and total bilirubin >2×ULN or international normalised ratio >1.5 (i.e. Hy's law cases), (d) ALT or AST >3×ULN with the appearance of fatigue, nausea, vomiting, right upper quadrant pain or tenderness, fever, rash, and/or eosinophilia (>5%); Any life-threatening (grade 4) clinical event, including anaphylaxis, related to the investigational product as agreed upon consultation with the medical monitor; Recurrent severe (grade 3) hypersensitivity reaction related to the investigational product as agreed upon consultation with the medical monitor; Recurrent severe (grade 3) infusion reaction related to investigational product as agreed upon consultation with the medical monitor; Grade 3 or higher neutropenia that did not improve to at least grade 2 within 5 days, as agreed upon consultation with the medical monitor; Receipt of a prohibited medication in the randomised, controlled period before day 15 or during the open-label period following consultation with the medical monitor; Non-compliance with the study protocol as judged by the investigator and/or the sponsor.

### Example 2—Clinical Trial Subject Enrollment Requirements and Criteria

Overview. Key inclusion criteria were: adults with a diagnosis of NMOSD^{2,17} and EDSS score ≤8.0, and a history of either at least one attack requiring rescue therapy (intravenous corticosteroids, intravenous immunoglobulin, and/or plasma exchange) within the year before screening or at least two attacks requiring rescue therapy in the 2 years before screening. AQP4-IgG seropositive and seronegative patients were eligible; seronegative participants had to meet the Wingerchuk 2006 criteria.¹⁷. There were no pre-planned recruitment targets with regard to AQP4-IgG serostatus. It was assumed that recruitment would reflect the known demographics of the patient population of approximately 80% seropositive, 20% seronegative.¹⁷ All participants provided written informed consent.

Sample size. The original sample size calculation concluded that 212 patients would need to be recruited to observe a required 67 attacks. This number of patients was calculated by assuming hazard rates of 1.5 per year and 1.0 per year for an attack in the placebo arm for seropositive and seronegative groups, respectively. These hazard rates were based on the observed attack rates observed in four open-label cohort studies (Bedi, et al. Impact of rituximab on relapse rate and disability in neuromyelitis optica. Mult Scler 2011; 17: 1225-30; Costanzi, et al. Azathioprine: tolerability, efficacy, and predictors of benefit in neuromyelitis optica. Neurology 2011; 77: 659-66; Jacob, et al. Treatment of neuromyelitis optica with rituximab: retrospective analysis of 25 patients. Muscle Nerve 2008; 39: 87-90; Kim, et al. Repeated treatment with rituximab based on the assessment of peripheral circulating memory B cells in patients with relapsing neuromyelitis optica over 2 years. Arch Neurol 2011; 68: 1412-20; Pittock, et al. Eculizumab in AQP4-IgG-positive relapsing neuromyelitis optica spectrum disorders: an open-label pilot study. Lancet Neurol 2013; 12: 554-62).

The study encountered challenges in recruitment and in obtaining the required number of neuromyelitis optica spectrum disorder attacks projected to achieve statistical significance. Most participating sites contributed one to three patients to the study and additional sites were activated during the 3 years of active recruitment. These challenges were expected because of the rarity of the disease and the lack of established diagnostic tools in parts of the world. A patient consumption effect was noticed, in which centres screened and enrolled existing eligible patients soon after site activation, and then recruitment activity dropped. The lower-than-expected and changing rate of attacks during the study required an increased target number of patients from 212 to 252, based on projections of the number of patients that would be required to achieve the required number of attacks.

An interim sample size re-assessment was planned to be executed before a planned futility analysis. The rate of neuromyelitis optica spectrum disorder attacks is not established in the literature, so this re-assessment was important to protect against the loss of statistical power in the seropositive cohort, as well as in the overall population due to not achieving the required number of events. Owing to a lower than expected attack rate and the possibility that the sample size assessment would be done first, and the rate subsequently changing with no further opportunity to increase the sample size, the protocol was amended in collaboration with the US Food and Drug Administration on 15 Dec. 2016. The pre-planned sample size re-assessment was removed, and the enrollment goal was changed to 252. This was determined by analysing the actual attack rate based on masked data from the first 78 patients who completed the randomised controlled period in this study. The attack status (attack/no attack) of the 78 patients was randomly sampled with different attack rates which gave an estimate of the number of attacks for the total sample size. This simulation process was repeated 10 000 times to give a distribution of the number of attacks for the total sample size, from which the probability of observing at least 67 attacks could be estimated. Based on the 78 completed patients, this procedure showed that with a sample size of 227 there was a 50% probability of reaching the required 67 adjudication-committee-determined attacks and with 252 patients there was a 90% probability of reaching the required 67 adjudication-committee-determined attacks. As such, the sample size of 252 patients was selected to give a high degree of confidence that 67 attacks would be observed in this study.

Statistical analysis—number needed to treat and multiplicity adjustment strategy. The primary endpoint was assessed by survival analysis, with Cox proportional hazards regression with placebo as the reference group, and treatment and serotype as explanatory factors. The proportional hazards model assumption was checked by visual inspection of the survival curves and by testing the assumption (p=0.1790); both checks suggest that the proportional hazard model assumption holds. Owing to the fact that primary endpoint is a time to event outcome, the number needed to treat was estimated from the survival probabilities for the VIB551 and placebo groups and the hazard ratio comparing the two groups, as discussed in Altman D G, Anderson P K. Calculating the number needed to treat for trials where the outcome is time to an event. BMJ 1999; 319: 1492-5. The time to event nature of the primary endpoint also meant that informative censoring was a possibility. Only seven participants out of a total of 230 (two from placebo and five from VIB551) did not complete the randomised controlled period. Given this and the magnitude of the treatment effect observed with 43 attacks during the randomised controlled period, the impact of potentially informative censoring was considered minimal.

For the key secondary endpoints, the odds ratio of EDSS score worsening was assessed using a logistic regression model with treatment, serostatus, and baseline score as explanatory variables and non-responder imputation. Least-squares mean differences in the change in low-contrast visual acuity binocular score were assessed using an analysis of covariance model with treatment, serostatus, and baseline Landolt C Broken Ring Chart binocular score as explanatory variables and last non-missing low-contrast visual acuity score. Rate ratios for the cumulative number of active MRI lesions and number of disease-related inpatient hospitalisations were assessed using negative binomial regression with treatment and serostatus as explanatory variables. Whole blood samples for quantification of B cells were collected according to the schedule of assessments; B-cell counts and immunophenotyping analyses of various B-cell subsets were done by flow cytometry performed at a central laboratory, with data reported descriptively. Treatment-emergent adverse events were summarised by system organ class and preferred terms using Medical Dictionary for Regulatory Activities version 21.0 and were reported descriptively.

The primary endpoint and the four secondary endpoints were considered to establish type 1 error control.

Primary endpoint: Time (in days) from day 1 to onset of an adjudication-committee-determined neuromyelitis optica spectrum disorder attack on or before day 197. The definition of an attack was the presence of a new symptom(s) or worsening of an existing symptom(s) related to neuromyelitis optica that met at least one of the protocol-defined criteria for a neuromyelitis optica spectrum disorder attack.

Four key secondary endpoints: 1. Worsening from baseline in EDSS score at last visit during the randomised, controlled period. The EDSS assessment in the study was performed by an independent and blinded assessor at each site using an electronic data capture system, developed by the University of Basel and Neurostatus GmBH, which contained an internal algorithm providing feedback to the assessor on inconsistencies in the EDSS assessment; 2. Change from baseline in low-contrast visual acuity binocular score measured by low-contrast Landolt C Broken Ring Chart at last visit during the randomised controlled period; 3. Cumulative total number of active MRI lesions (new gadolinium-enhancing or new/enlarging T2 lesions) during the randomised controlled period; 4. Number of neuromyelitis optica-related inpatient hospitalisations, with inpatient hospitalisation defined as more than an overnight stay.

Based on two populations of interest (seropositive and ITT populations), this resulted in testing 10 null hypotheses of no treatment effect. The pre-specified futility analysis was not required to be factored into type 1 error control, nor were the ongoing evaluations of the data monitoring committee. Both were independent of the primary and key secondary endpoints and have no effect on these analyses.

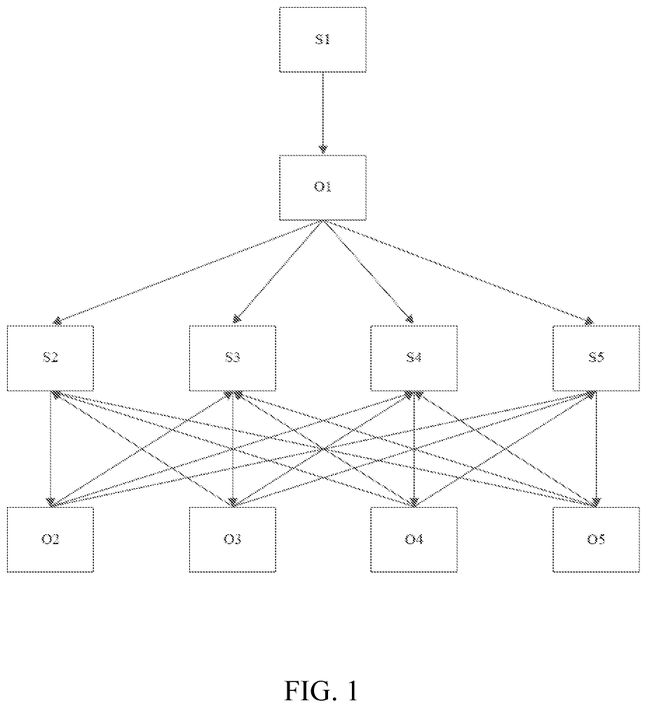

The multiplicity adjustment strategy was based on the Bonferroni-based chain procedure. Each hypothesis is represented by a rectangular box in FIG. 1. The connections between the hypotheses are shown using arrows. A solid arrow is used to define the decision path after a hypothesis is rejected (e.g. the hypothesis O1 is tested if, and only if, the hypothesis S1 is rejected).

The Bonferroni-based chain procedures are characterised by two rules: 1. The α allocation rule specifies the initial distribution of the type 1 error rate among the null hypotheses according to the relative importance of the null hypotheses; 2. The α propagation rule determines the process of re-distributing the available type 1 error rate among the non-rejected null hypotheses after each rejection.

In terms of α allocation, the null hypothesis S1 receives the initial weight of 1 (i.e. it is tested at the full α=0 05) and the other null hypotheses receive zero weights. The α propagation rule states that if the null hypothesis O1 is rejected, the available type 1 error rate will be split equally among the null hypotheses S2, S3, S4, and S5 (one-quarter of the available type 1 error rate will be allocated to each null hypothesis). The α allocation and α propagation rules uniquely define the Bonferroni-based chain procedure and the associated multiplicity-adjusted p values can be computed using algorithm 2 given in Bretz, et al., 2009.

In N-MOmentum, the primary endpoint was hierarchically tested at α=0.05, first in the AQP4-IgG seropositive cohort, and, if significant, in the overall ITT population. The reason for this approach is that despite the use of the Wingerchuck 2006 criteria and the role of the independent eligibility committee, the possibility of uncertainty remained regarding the precise nature of the AQP4-IgG seronegative cohort and how they might respond to B-cell depletion. As AQP4-IgG seropositive patients represent the majority of people with neuromyelitis optica, and the direct relationship between AQP4 and the B-cell-related mechanism of action is clear, it was decided to test the primary endpoint statistically in AQP4-IgG seropositive participants first, before then testing the overall cohort. In this manner, the intent was to ensure that the maximum amount of clinically relevant data was gathered while mitigating any potential impact of a differential effect in AQP4-IgG seronegative participants.

If the primary endpoint was met in the ITT population, then all key secondary hypotheses were tested in the same manner, with each secondary hypothesis initially tested based on the chain procedure at α=0.0125. If the null hypothesis for a secondary endpoint was rejected across both the seropositive and overall populations, the type 1 error saved was propagated equally to other non-rejected sets of secondary null hypotheses. P values are presented in the main text following multiplicity adjustment as described above with α=0.05.

Inclusion criteria. In order to be included in the trial, subject were required to meet all of the following criteria: (1) Age 18 years or above at the time of screening; (2) Written informed consent and any locally required authorization (e.g., Health Insurance Portability and Accountability Act [HIPAA] in the United States of America (USA), European Union [EU] Data Privacy Directive in the EU) obtained from the subject/legal representative prior to performing any protocol-elated procedures, including screening evaluations; (3) One of the following: (a) Positive serum anti-AQP4-IgG result at screening (verified by the allocated central laboratory) and a documented history of one or more NMO/NMOSD acute relapses that required rescue therapy within the last year, or 2 or more NMO/NMOSD acute relapses that required rescue therapy within 2 years prior to screening; OR (b) Negative serum anti-AQP4-IgG result at screening (verified by the allocated central laboratory) without evidence of brain lesion consistent with MS and meets the clinical criteria for NMO according to Wingerchuk et al, 2006 and a documented history of one or more NMO acute relapses that required rescue therapy within the last year, or 2 or more NMO acute relapses that required rescue therapy within 2 years prior to screening. Note that data from AQP4-IgG seronegative subjects will be reviewed by an independent eligibility committee (see Section 4.2.1.1) to confirm eligibility (n the event that a subject has not received rescue therapy for a relapse due to a misdiagnosis or mismanagement of symptoms at a practice or medical center outside of the investigator's control, the subject may still be eligible for the study if, following a review of the relapse data, the medical monitor and investigator are satisfied that the subject experienced a genuine relapse); (4) Subjects who have had a relapse immediately prior to screening must have at least 4 weeks in which their relapse symptoms are stable or improving prior to randomization; (5) Expanded Disability Status Scale score at randomization less than or equal to 7.5. A score of 8.0 may be eligible if the investigator and medical monitor assess that the subject is reasonably able to participate in the study; (6) Females of childbearing potential who are sexually active with a nonsterilized male partner must use a highly effective method of contraception (subjects in the Czech Republic only must use 1 additional method of contraception) from screening, and must agree to continue using such precautions for 6 months after the final dose of investigational product; cessation of contraception after this point should be discussed with a responsible physician. Periodic abstinence, the rhythm method, and the withdrawal method are not acceptable methods of contraception, (a) Females of childbearing potential are defined as those who are not surgically sterile (ie, bilateral tubal ligation, bilateral oophorectomy, or complete hysterectomy) or those who are not postmenopausal (per International Council for Harmonisation [ICH] M3(R2) 11.2: defined as 12 months with no menses without an alternative medical cause), (b) A highly effective method of contraception is defined as one that results in a low failure rate (ie, less than 1% per year) when used consistently and correctly. The acceptable highly effective methods of contraception are described in Table 4; (7) Nonsterilized males who are sexually active with a female partner of childbearing potential must use a male condom plus spermicide (subjects in the Czech Republic only must use 1 additional method of contraception) (see Table 4) from Day 1 for 3 months after receipt of the final dose of investigational product. Because male condom and spermicide is not a highly effective contraception method, it is strongly recommended that female partners of male subjects to also use a highly effective method of contraception throughout this period; (8) Sterilized males, without the appropriate post-vasectomy documentation on the absence of sperm in the ejaculate, who are sexually active with a female partner of childbearing potential must use a condom and spermicide from Day 1 for 3 months after receipt of the final dose of investigational product.

Exclusion criteria. Any of the following would exclude a subject from participation in the study: (1) Any condition that, in the opinion of the investigator, would interfere with the evaluation or administration of the investigational product or interpretation of subject safety or study results; (2) Concurrent/previous enrollment in another clinical study involving an investigational treatment within 4 weeks or 5 published half-lives of the investigational treatment, whichever is the longer, prior to randomization; (3) An estimated glomerular filtration rate (GFR) of <60 mL/minute; (4) Lactating or pregnant females or females who intend to become pregnant anytime from signing the ICF through the study plus 6 months following last dose of investigational product; (5) Known history of allergy or reaction to any component of the investigational product formulation or history of anaphylaxis following any biologic therapy; (6) Evidence of alcohol, drug, or chemical abuse, or a recent history of such abuse <1 year prior to randomization; (7) Major surgery within 8 weeks prior to signing the ICF, or elective surgery planned from screening through the duration of the RCP of the study; (8) Spontaneous or induced abortion, still or live birth, or pregnancy ≤4 weeks prior to signing the ICF; (9) Subjects who are unable to undergo an MRI scan (eg, hypersensitivity to Gd-containing MRI contrast agents, implanted pacemakers, defibrillators, or other metallic objects on or inside the body that limit performing MRI scans); (10) At screening (one repeat test may be conducted to confirm results prior to randomization within the same screening period), any of the following—(a) Aspartate transaminase (AST) >2.5×upper limit of normal (ULN), (b) Alanine transaminase (ALT) >2.5×ULN, (c) Total bilirubin >1.5×ULN (unless due to Gilbert's syndrome), (d) Platelet count <75,000/μL (or <75×109/L), (e) Hemoglobin <8 g/dL (or <80 g/L), (f) Glycosylated hemoglobin (I IbAlc) >8% at screening (subjects with diabetes only), (g) CD19. B cell counts below the lower limit of normal (LLN) according to the central laboratory. Any of the following related to concomitant medications would exclude a subject from participation in the study: (11) Receipt of the following at any time prior to randomization—(a) Alemtuzumab; (b) Total lymphoid irradiation; (c) Bone marrow transplant; (d) T-cell vaccination therapy; (12) Receipt of rituximab or any experimental B-cell depleting agent within the 6 months prior to screening, unless the subject has B-cell counts above the LLN according to the central laboratory; (13) Receipt of IVIG within 1 month prior to randomization; (14) Receipt of any of the following within 3 months prior to randomization—(a) Natalizumab (Tysabri®), (b) Cyclosporin, (c) Methotrexate, (d) Mitoxantrone, (e) Cyclophosphamide, (f) Tocilizumab, (g) Eculizumab; or (15) Severe drug allergic history or anaphylaxis to two or more food products or medicine (including known sensitivity to acetaminophen/paracetamol, diphenhydramine or equivalent antihistamine, and methylprednisolone or equivalent glucocorticoid). Any of the following criteria related to NMO and other diseases would exclude a subject from participation in the study: (16) AQP4-IgG seronegative subjects with a brain MRI abnormality that meets the diagnostic criteria for MS (MRIs taken at screening will be assessed centrally); (17) Uncontrolled hypertension as indicated by the treating physician and/or principal investigator; (18) Any concomitant disease other than NMO that required treatment with oral or IV steroids at doses >20 mg/day for >21 days within the 6 months prior to screening; (19) Any subjects diagnosed with a concurrent autoimmune disease that is either uncontrolled or requires the use of disease-modifying agents or immunosuppressive agents. Any of the following criteria related to infection and malignancy risk factors would exclude a subject from participation in the study: (20) Receipt of any of the following—(a) Any live or attenuated vaccine within 3 weeks prior to Day 1 (administration of killed vaccines is acceptable, the Sponsor recommends that investigators ensure all subjects are up to date on required vaccinations prior to study entry), (b) Bacillus of Calmette and Guerin (BCG) vaccine within 1 year of signing the ICF, (c) Blood transfusion within 4 weeks prior to signing the ICF; (21) Clinically significant serious active or chronic viral or bacterial infection that requires; treatment with anti-infectives, hospitalization, or, in the investigator's opinion, represents an additional risk to the subject, within 60 days prior to randomization; (22) Known history of a primary immunodeficiency (congenital or acquired) or an underlying condition such as human immunodeficiency virus (HIV) infection or splenectomy that predisposes the subject to infection; (23) At screening (one repeat test may be conducted to confirm results prior to randomization within the same screening period), any of the following—(a) Total Ig <600 mg/dL, (b) Absolute neutrophil count <1200 cells/μL, (c) CD4 T lymphocyte count <300 cells/μL; (24) Confirmed positive test for hepatitis B/hepatitis C serology at screening—(a) Hepatitis B surface antigen positive, (b) Hepatitis B core antibody positive with negative hepatitis B surface antibody, (c) Hepatitis C antibody positive; (25) Subjects with a positive QuantiFERON®-TB Gold test, unless an appropriate course of anti-tuberculosis (TB) treatment has been documented. Subjects with an indeterminate result may be eligible if a chest x-ray shows no evidence of TB and there is no evidence of latent TB; (26) History of cancer, apart from squamous cell or basal cell carcinoma of the skin treated with documented success of curative therapy >3 months prior to randomization.

### Example 3—Clinical Trial Protocol

Screening period. Subjects with a diagnosis of NMO/NMOSD were screened over a 28-day period to establish their eligibility to participate in the study based on the inclusion and exclusion criteria. All subjects who fulfilled eligibility criteria were randomized into the study.

Randomization. The subjects were randomized into the study in a 3:1 ratio to receive intravenously VIB551 (30 mg) or placebo as described in Table 1. Randomization occurred on Day 1 and was stratified by AQP4-IgG serostatus (in a ratio of approximately 80:20 seropositive and seronegative subjects, respectively) and by region (Japan vs non-Japan).

TABLE 1
Randomized-controlled period treatment regimen

| Treatment | |
|---|---|
| Arm | Treatment Regimen |
| 1 | 300 mg intravenous MEDI-551 |
| | on Day 1 and Day 15 |
| 2 | intravenous Placebo on Day 1 |
| | and Day 15 |

Randomized-controlled period (Day 1 to Day 197). Following randomization on Day 1, the subjects were treated with VIB551 or placebo on Day 1 and Day 15. An oral corticosteroid course was initiated on Day 1 (prednisone 20 mg/day or equivalent oral glucocorticoid) and continued until Day 14. Tapering of the oral corticosteroids occurred from Day 15 to Day 21 (for prednisone: 15 mg prednisone on Day 15, 10 mg prednisone on Day 16, 7.5 mg prednisone on Day 17, 5 mg prednisone on Days 18 and 19, and 2.5 mg prednisone on Days 20 and 21). By Day 21, tapering was completed. The rationale for use of oral corticosteroids (prednisone 20 mg/day or equivalent oral glucocorticoid) for the first 14 days (with a 1-week taper) was to provide prophylaxis against a neuromyelitis optica spectrum disorder attack for the period wherein the pharmacodynamic effect of VIB551 was not expected; a period of approximately 2-4 weeks is required for maximal B-cell depletion to occur.

During the randomized controlled period, the subjects were followed at scheduled study visits and by telephone interview. The duration of the randomized control period for each subject was planned to be 197 days. All subjects who completed the randomized control period without experiencing an NMO/NMOSD attack were given the option to enter the open-label period.

Open-label period. Subjects were given the option to enter the open label period if they: (1) completed 197 days of the randomized control period; (2) experienced an adjudication committee-determined NMO/NMOSD attack during the randomized control period; (3) were in the randomized control period at the time when 67 adjudication committee-determined NMO/NMOSD attacks had occurred; or (4) were in the randomized control period when enrollment was discontinued upon recommendation of the DMC based on evidence of efficacy and safety.

Patients who discontinued the randomised controlled period for reasons other than an adjudicated attack or the occurrence of 67 adjudicated attacks were not eligible for the open-label period. Reasons for patients not entering the open-label period were captured. These patients were then followed up for safety in the safety follow-up period.

Upon entering the open-label period for one of the four reasons outlined above, patients received VIB551 300 mg every 26 weeks; however, patients randomised to placebo during the randomised controlled period received an additional 300 mg dose on day 15 of the open-label period to maintain a total initial dose of 600 mg. Table 2 provides the open-label period treatment regimens.

TABLE 2
Open-label Period Treatment Regimen

| Treatment | |
|---|---|
| Arm in | |
| RCP | Treatment Regimen |
| 1 | 300 mg IV MEDI-551 on OLP Day 1, masked |
| | IV placebo OLP Day 15, then 300 mg IV |
| | MEDI-551 Q26W thereafter^{a} |
| 2 | 300 mg IV MEDI-551 on OLP Day 1, masked |
| | 300 mg IV MEDI-551 on OLP Day 15, then |
| | 300 mg IV MEDI-551 Q26W thereafter^{a} |

IV = intravenous;
OLP = Open-label Period;
Q26W = every 26 weeks;
RCP = Randomized-controlled Period;
SFP = Safety Follow-up Period.
^{a}The OLP will continue for a minimum of 1 year after the last
subject enters and a maximum of 3 years (after the last subject
enters), or until regulatory approval for MEDI-551 in the
participating country, or until the Sponsor discontinues
development of MED1-551 in this indication, whichever
occurs first. Subjects can choose to exit the OLP at any time
for any reason, including seeking alternative treatment options,
at which point they will enter the SFP (unless consent is withdrawn).

During the open-label period, patients were followed up at scheduled study visits and continued to receive VIB551 therapy for a maximum of 3 years (after the last patient entered), until regulatory approval for VIB551 in each participating country or until discontinuation of the development of VIB551 by the sponsor in this indication, whichever occurred first. Patients were followed up for attacks in the same fashion as in the randomised, controlled period and events were centrally adjudicated.

Patients could choose to exit the open-label period at any time for any reason, including seeking alternative treatment options, at which point they entered the safety follow-up period (unless consent was withdrawn).

Safety follow-up period. The safety follow-up period started when a patient prematurely discontinued from the randomised controlled or open-label periods. The length of the safety follow-up period was determined by the time elapsed from the time of the last dose to the time of the premature discontinuation, to complete a total of 52 weeks. During the safety follow-up period, patients were monitored for adverse/serious adverse events, B-cell levels, anti-drug antibodies, and immunoglobulin levels. Patients could receive standard treatment for their condition, at the discretion of the investigator.

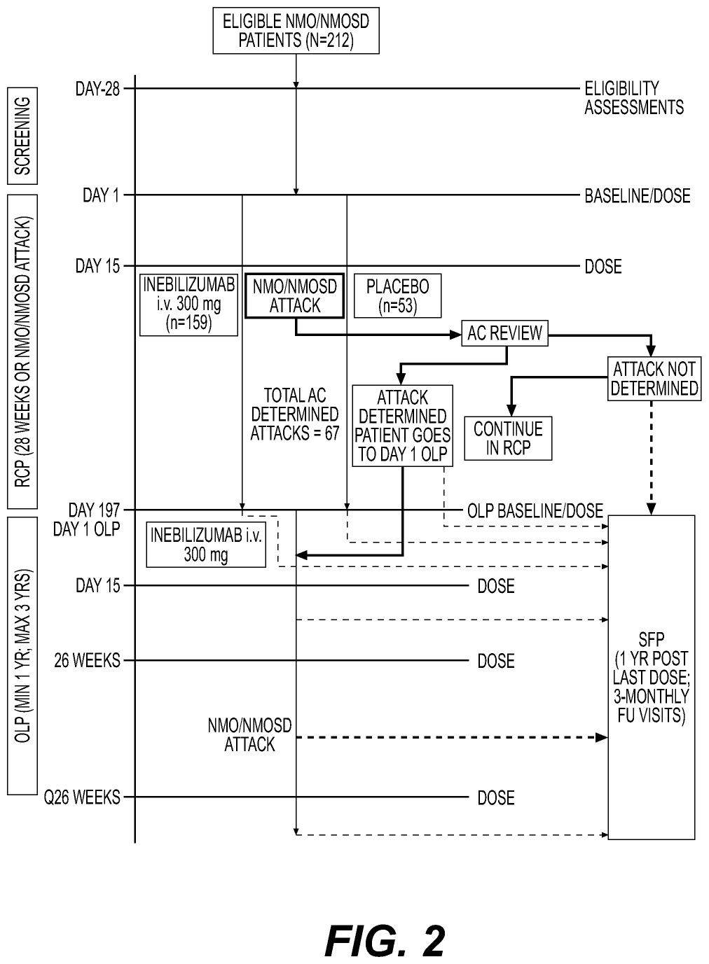

An overall study design flow diagram is provided at FIG. 2.

### Example 4—Patient Monitoring During Clinical Trial Treatment Period

Randomized-controlled Treatment Period. Following a screening period (<56 days), eligible participants were randomised by a central interactive voice/web response system 3:1 (permuted block randomisation scheme) to intravenous VIB551 300 mg or placebo, respectively, administered on days 1 and 15. Participants, investigators, sponsor, adjudication committee, and staff involved in patient treatment or clinical evaluation, including the EDSS rater, were masked/blinded to the treatment received, with VIB551 and placebo indistinguishable in appearance.

Table 3 shows all procedures that were to have be conducted during the Randomized-controlled treatment period. Patient-reported outcomes were to have been done first followed by all other assessments/procedures in an order determined by the site. If a subject decided to enroll into the open-label period, the subject followed the enrollment procedures of the open-label period in Table 4.

TABLE 3
Schedule of Randomized-controlled Treatment Period Study Procedures

| | Randomised controlled period | | | | | | | | |
|---|---|---|---|---|---|---|---|---|---|
| | Week | | | | | | | | |
| | | | | | | | | | 28/ |
| | 0 | 1 | 2 | 4 | 8 | 12 | 16 | 22 | EDV |
| | Day | | | | | | | | |
| | 1†† | 8 | 15 | 29 | 57 | 85 | 113 | 155 | 197 |
| | Visit window | | | | | | | | |
| | | ±1 d | ±3 d | ±3 d | ±3 d | ±3 d | ±3 d | ±3 d | ±7 d |
| | Procedure/visit number | | | | | | | | |
| | 2 | 3 | 4 | 5 | 6 | 7 | 8 | 9 | 10 |
| Verify eligibility criteria | X | | | | | | | | |
| SF-36v2* | X | | | | | X | | | X |
| Pain NRS | X | | | X | X | X | X | X | X |
| Modified Rankin Scale | X | | | X | X | X | X | X | X |
| Medical and disease history (including | X | | | | | | | | |
| smoking and alcohol history) | | | | | | | | | |
| C-SSRS (Since Last Visit version)** | X | X | X | X | X | X | X | X | X |
| Physical/neurological examination | X | X | X | X | X | X | X | X | X |
| Body weight | X | | | | | X | | | X |
| Vital signs | X | X | X | X | X | X | X | X | X |
| ECG | X | | | | | | | | X |

Blood tests

| | | | | | | | | | |
|---|---|---|---|---|---|---|---|---|---|
| Haematology and serum chemistry | X | X | X | X | X | X | X | X | X |
| VIB551 concentration (PK) | X † | X | X † | X | X | X | X | X | X |
| VIB551 ADA | X | | | X | | X | | | X |
| Total Ig, IgM, IgG, IgA, IgE | — | | | | | X | | | X |
| Whole blood for flow cytometry (B-cell | X | X | X | X | X | X | X | X | X |
| count and cell subsets) | | | | | | | | | |
| Serum for AQP4-IgG assay (titres) | X | | X | X | X | X | | | X |
| Whole blood for gene expression | X | | X | X | X | X | X | | X |
| Whole blood for DNA sample (optional) | X | | | | | | | | |
| Serum for exploratory biomarkers | X | | X | X | X | X | X | X | X |
| Vaccination titres (tetanus)‡‡ | X | | | | | | | | X |

Urine tests

| | | | | | | | | | |
|---|---|---|---|---|---|---|---|---|---|
| Urine HCG (women of childbearing potential | X | | X | | | | | | X |
| only) (pre-i.v. dose only)‡ | | | | | | | | | |
| Urinalysis | X | X | X | X | X | X | X | X | X |
| Assess patient for new/worsening symptoms | X | X | X | X | X | X | X | X | X |
| related to neuromyelitis optica spectrum | | | | | | | | | |
| disorder§, ¶ | | | | | | | | | |
| Independent EDSS/FSS administration** | X | | | | | X | | | X |
| Independent ophthalmology examination | X | | | | | X | | | X |
| (high-/low-contrast visual acuity test, | | | | | | | | | |
| RAPD assessment) | | | | | | | | | |
| Neuroaxis MRI (including optic nerve) | | | | | | | | | X |
| Randomise patients in IVRS | X | | | | | | | | |
| Investigational product administration | X | | X §§ | | | | | | |
| Dispense/monitor low-dose steroids | X | X | X | X | | | | | |
| Assessment of AEs/SAEs (including | X | X | X | X | X | X | X | X | X |
| infusion reactions) | | | | | | | | | |
| Concomitant medications | X | X | X | X | X | X | X | X | X |
| Dispense and review patient HCRU | X | X | X | X | X | X | X | X | X |
| diaries ∥ | | | | | | | | | |

| | |
|---|---|
| Follow-up telephone call | Telephone calls every 2 weeks will commence from day 43 and |
| | continue for the duration of the randomised controlled period, |
| | except for study visit weeks |

Note:
all procedures performed before randomisation.
*SF-36v2 4-week recall used at all time points. The 1-week recall version was also used for an attack.
†Pharmacokinetic blood samples collected pre-dose and approximately 15 minutes post-dose (±5 minutes) after completion of VIB551 or placebo administration.
‡Urine HCG followed up for ≤26 weeks following discontinuation.
§If the attack was before day 1 and patient not randomised, the visit could not continue. The patient had to be treated for the attack as required, screen failed, and then re-assessed for eligibility once the patient's condition was stable.
¶In the event that an attack was suspected, assessment procedures had to be followed.
∥ Patient diaries distributed to aid in patient recollection of HCRU events.
**If the EDSS and the C-SSRS were performed by the same person, the EDSS had to be done first.
††If necessary, the randomisation visit could be split over 2 days. In this case, randomisation and investigational product administration had to occur on the second day.
‡‡Tetanus vaccine titres tested on day 1 for all patients. Patients with a negative result did not continue to be tested. Patients who tested positive continued to be tested at all specified vaccination titre time points.
§§ In the event that the second dose of investigational product was delayed due to medical/safety reasons, dosing was discussed with the medical monitor before administration of investigational product.
ADA, anti-drug antibody; AE, adverse event; AQP4-IgG, aquaporin-4 immunoglobulin G; C-SSRS, Columbia-Suicide Severity Rating Scale; d, day(s); EDSS, Expanded Disability Status Scale; EDV, early discontinuation visit; ECG, electrocardiogram; FSS, functional systems score; HCG, human chorionic gonadotropin; HCRU, healthcare resource utilisation; IgA/E/G/M, immunoglobulin A/E/G/M; i.v., intravenous; IVRS, interactive voice response system; MRI, magnetic resonance imaging; NRS, numeric rating scale; PK, pharmacokinetic; RAPD, relative afferent pupillary defect; SAE, serious adverse event; SF-36v2, 36-item Short-Form Health Survey, version 2.

Open-label Period. All procedures to be conducted during the open-label period are shown in Table 4. Patient-reported outcomes were done first followed by all other assessments/procedures in an order determined by the site. For subjects completing Day 197 of the randomized control period, Day 1 of the open-label period should be the same day; however, it may be delayed for up to 14 days (procedure do not need to be repeated). Subjects are not permitted to enter the open label period after 14 days, unless there is a compelling reason that is discussed with, and agreed to by the medical monitor, in which case a short extension may be granted.

Subjects who experienced an adjudication committee-determined NMO/NMOSD attack during the randomized control period were to enter the open-label period within 28 days of the site receiving confirmation of the attack from the adjudication committee. Procedures that were done during the last visit could be used for Day 1 of the open-label period if this occurs within 14 days from the Assessment Visit. Otherwise, procedures required for open-label period Day 1 should be performed.

Subjects who were in the randomized control period at the time the 67^{th} adjudication committee-determined attack occurred, or when enrollment was discontinued upon recommendation of the independent DMC based on evidence of efficacy and safety, and wished to enroll in the open label period, should have done so as soon as possible, preferably within 14 days. Consultation with the medical examiner was advised in cases where the transition to the open label period is not completed within 14 days.

Administration of VIB551 began at open label period day 1.

The open label period was to be continued for a minimum of 1 year after the last subject enters and a maximum of 3 years (after the last subject enters), or until regulatory approval for VIB551 in the participating country, or until VIB551's development in NMO/NMOSD is discontinued.

| | Open-label Period | | | | | | |
|---|---|---|---|---|---|---|---|
| | Week | | | | | | |
| | | | | | | 39, then | 52, then |
| | 0 | 2 | 4 | 13 | 26 | Q26 W | Q26 W/EDV |
| | Day | | | | | | |
| | 1 | 15 | 29 | 92 | 183 | 274+ | 365+ |
| | Visit Window | | | | | | |
| | (+14 d from | | | | | | |
| | Day 197 RCP | | | | | | |
| | or +28 d AC | | | | | | |
| | Determination) | ±3 d | ±3 d | ±14 d | ±7 d | ±14 d | ±7 d |
| | Procedure/Visit Number | | | | | | |
| | | | | | | 6, 8, | 7, 9, |
| Study Period | 1 | 2 | 3 | 4 | 5 | 10, etc. | 11, etc. |
| Written informed consent | X | | | | | | |
| SF-36v2 Health Survey | | | | X | X | X | X |
| Pain NRS | | | X | X | X | X | X |
| Modified Rankin Scale | | | X | X | X | X | X |
| Physical/neurological examination | X^{a} | X | X | X | X | X | X |
| C-SSRS (“Since Last Visit” version)^{h} | X^{a} | X | X | X | X | X | X |
| Vital signs | X^{a} | X | X | X | X | X | X |

Collect blood for:

| | | | | | | | |
|---|---|---|---|---|---|---|---|
| Hematology and serum chemistry | X^{a} | X | X | X | X | X | X |
| MEDI-551 ADA | X^{a} | | | X | X | X | X |
| Total Ig, IgM, IgG, IgA, IgE | X^{a} | | | X | X | X | X |
| Whole blood for flow cytometry (B-cell | | X | X | X | X | X | X |
| count and cell subsets) | | | | | | | |
| Serum for AQP4-IgG assay (titers) | X^{a} | | X | X | X | | X |
| Whole blood for gene expression | | | X | X | X | | X |
| Serum for exploratory biomarkers | | X | X | X | X | X | X |
| Vaccination titers (tetanus)^{f} | | | | X | X | | X |

Collect Urine For:

| | | | | | | | |
|---|---|---|---|---|---|---|---|
| Urine HCG (females with childbearing | X^{b} | X | | | X | | X |
| potential only; pre-IV dose only) | | | | | | | |
| Urinalysis | X^{a} | X | X | X | X | X | X |
| Assess subject for new/worsening symptoms | X^{b} | X | X | X | X | X | X |
| related to NMO/NMOSD^{c} | | | | | | | |
| Independent EDSS/FSS administration^{h} | — | | | X | X | X | X |
| Independent ophthalmology examination (high-/ | | | | X | X | X | X |
| low-contrast visual acuity test, RAPD assessment) | | | | | | | |
| Neuroaxis MRI (including optic nerve) | (X)^{d} | | | | | | X^{a} |
| Investigational product administration | X^{b} | Xⁱ | | | X | | X^{g} |
| Assessment of AEs/SAEs (including infusion | X^{b} | X | X | X | X | X | X |
| reactions) | | | | | | | |
| Concomitant medications | X^{b} | X | X | X | X | X | X |
| Dispense and review subject HCRU diary entries^{f} | X^{b} | X | X | X | X | X | X |

AC = Adjudication Committee;
ADA = anti-drug antibody;
AE = adverse event;
AQP4-IgG = autoantibodies against aquaporin-4;
C-SSRS = Columbia-Suicide Severity Rating Scale;
d = day(s);
EDSS = Expanded Disability Status Scale;
EDV = Early Discontinuation Visit;
FSS = Functional Systems Scores;
HCG = human chorionic gonadotropin;
HCRU = Healthcare Resource Utilization;
Ig A/E/G/M = immunoglobulin A/E/G/M;
IV = intravenous;
MRI = magnetic resonance imaging;
NMO/NMOSD = neuromyelitis optica/neuromyelitis optica spectrum disorders;
NRS = numeric rating scale;
OLP = Open-label Period;
Q26 W = every 26 weeks;
RAPD = relative afferent pupillary defect;
RCP = Randomized-controlled Period;
SAE = serious adverse event;
SF-36v2 = Short Form-36 Health Survey, version 2
^{a}For subjects who enter the OLP prior to Day 197 of the RCP due to an AC-determined NMO/NMOSD attack and 14 days have elapsed since the Assessment Visit, all procedures listed for Day 1 should be repeated. For subjects completing Day 197 of the RCP, Day 1 of the OLP should be the same day; however, it may be delayed for up to 14 days and procedures do not need to be repeated.
^{b}Conduct for all subjects.
^{c}In the event that an NMO/NMOSD attack is suspected, procedures in Section 4.2.3 should be followed.
^{d}If the subject experienced an NMO/NMOSD attack during the RCP, any part of the neuroaxis MRI that was not done at the time of the Assessment Visit should be conducted at Day 1 of the OLP, otherwise it is not required.
^{e} An MRI will be conducted yearly.
^{f}Subject diaries will be distributed to aid in subject recollection of HCRU events.
^{g}If an EDV is being conducted, investigational product will not be given.
^{h}If the EDSS and the C-SSRS are performed by the same person, the EDSS must be done first.
ⁱIn the event that the second dose of investigational product is delayed due to a medical/safety reason, dosing must be discussed with the medical monitor prior to administration of investigational product.
^{j} Tetanus vaccine titers will only be tested in the OLP for subjects who tested positive at Day 1 of the RCP.

Assessment Visits for subjects experiencing new or worsening symptom(s) potentially related to NMO/NMOSD. As assessment visit should have been scheduled as soon as possible and within 72 hours of symptom report. Table 5 shows all study procedures to be conducted at Assessment Visits. Procedures to determine whether the symptoms are related to NMO/NMOSD should be conducted first, followed by procedures for determination of an NMO/NMOSD attack. Table 6 shows the criteria used for an NMO/NMOSD attack, with criteria-based severity. All clinical assessments needed to have been documented with time and date, and the order of the assessments should have been determined by the nature of the suspected attack (e.g., EDSS before Independent ophthalmology in case of myelitis symptoms). An MRI of all domains should have been performed as part of an Assessment Visit and could have been done at any time during the Assessment Visit. The order of the MRI domains performed could have been determined by the nature of the suspected attack. MRI images/study report should not have been reviewed by the principal investigator unless a specific criterion for an attack requires review of the MRI. In such cases review of the MRI must have been done after the review of all relevant clinical assessment data (e.g., EDSS in the case of myelitis/brainstem/brain symptoms or ophthalmology in case of optic neuritis symptoms) was completed. Assessments should have been concluded as soon as possible, but should not have extended beyond 4 days from Day 1 of the Assessment visit.

If the investigator had determined that the new or worsening symptom(s) was not related to NMO/NMOSD, then the independent assessments (EDSS, Independent ophthalmology, and MRI) were not required.

TABLE 5
Schedule of Study Procedures for the Assessment Visit

| | Assessment |
|---|---|
| Procedure | Visit^{a} |
| Pain NRS | X |
| Vital signs | X |
| Modified Rankin Scale | X |
| Physical/neurological examination | X |
| Collect blood for: | |
| Hematology and serum chemistry | X |
| MEDI-551 ADA | X |
| Total Ig, IgM, IgG, IgA, IgE | X |
| Whole blood for flow cytometry (B-cell count and | X |
| cell subsets) | |
| Serum for AQP4-IgG assay (titers) | X |
| Whole blood for gene expression | X |
| Serum for exploratory biomarkers | X |
| Collect urine for urinalysis | X |
| Procedures for determination of an NMO/NMOSD | |
| attack: | |
| Independent ophthalmology examination (high-/low- | X |
| contrast visual acuity test, RAPD assessment) | |
| Independent EDSS/FSS administration^{c} | X |
| Neuroaxis MRI scan^{b} | X |
| SF-36 (1-week recall version) | X |
| C-SSRS (″Since Last Visit″ version)′ | X |
| ECG | X |
| Review data generated against protocol defined | X |
| attack criteria and create a narrative of the | |
| Assessment Visit findings | |
| Assessment of AEs and SAEs | X |
| Concomitant medications | X |
| Review subject diary entries | X |

ADA = anti-ding antibody;
AE = adverse event;
AQP4-IgG = autoantibodics against aquaporin4;
C-SSRS = Columbia Suicide Severity Rating Scale;
ECG = electrocardiogram;
EDSS = Expanded Disability Status Scale;
FSS = Functional Systems Scores;
Ig A/E/G/M = immunoglobulin A/E/G/M;
MRI = magnetic resonance imaging;
NMO/NMOSD = neuromyelitis optica/neuromyelitis spcctrum disorders;
NRS = numeric rating scale;
RAPD = relative afferent pupillary defect;
SAE = serious adverse event;
SF-36 = Short Form-36 Health Survey

TABLE 6
Protocol-defined-criteria for an attack with criteria-based severity

| Example symptoms | | | |
|---|---|---|---|
| of a neuromyelitis | | | |
| optica spectrum | | | |
| disorder attack* | Attack type† | Protocol-defined attack criteria | |
| Blurred vision | ON | 1. | >15-character drop in high-contrast Landolt C Broken Ring Chart from |
| Loss of vision | | | last visit as measured in a previously affected eye and no other |
| Eye pain | | | ophthalmological explanation |
| | | 2. | Reduction of ≥ 2 steps‡ in CF to NLP from last visit as measured in a |
| | | | previously affected eye and no other ophthalmological explanation |
| | | 3. | Reduction of ≥ 7 characters in low-contrast Landolt C Broken Ring |
| | | | Chart from last visit as measured in either eye alone (monocular) AND |
| | | | a new RAPD in affected eye |
| | | 4. | Reduction of ≥ 7 characters in low-contrast Landolt C Broken Ring |
| | | | Chart from last visit as measured in either eye alone (monocular) AND |
| | | | loss of a previously documented RAPD in fellow eye |
| | | 5. | Reduction of ≥ 5 characters in high-contrast Landolt C Broken Ring |
| | | | Chart from last visit as measured in either eye alone (monocular) AND |
| | | | a new RAPD in affected eye |
| | | 6. | Reduction of ≥ 5 characters in high-contrast Landolt C Broken Ring |
| | | | Chart from last visit as measured in either eye alone (monocular) AND |
| | | | loss of a previously documented RAPD in fellow eye |
| | | 7. | Reduction of ≥ 1 step§ in CF to NLP from last visit as measured in a |
| | | | previously affected eye AND a new RAPD in affected eye |
| | | 8. | Reduction of ≥ 1 step§ in CF to NLP from last visit as measured in a |
| | | | previously affected eye AND loss of a previously documented RAPD |
| | | | in fellow eye |
| | | 9. | Reduction of ≥ 7 characters in low-contrast Landolt C Broken Ring |
| | | | Chart from last visit as measured in either eye alone (monocular) AND |
| | | | a new Gd-enhancing or new/enlarging T2 MRI lesion in the |
| | | | corresponding optic nerve¶ |
| | | 10. | Reduction of ≥ 5 characters in high-contrast Landolt C Broken Ring |
| | | | Chart from last visit as measured in either eye alone (monocular) AND |
| | | | a new Gd-enhancing or new/enlarging T2 MRI lesion in the |
| | | | corresponding optic nerve¶ |
| | | 11. | Reduction of ≥ 1 step§ in CF to NLP from last visit as measured in a |
| | | | previously affected eye AND a new Gd-enhancing or new/enlarging |
| | | | T2 MRI lesion in the corresponding optic nerve¶ |
| Deep or radicular | Myelitis∥ | 12. | Worsening of ≥ 2 points in at least one of the relevant (pyramidal, |
| pain | | | bladder/bowel, sensory) FSS compared with last visit |
| Extremity | | 13. | Worsening of ≥ 1 point in EDSS score compared with last visit if |
| paraesthesia | | | previous EDSS score ≥ 5.5 |
| Weakness | | 14. | Worsening of ≥ 1 point in at least two of the relevant (pyramidal, |
| Sphincter | | | bladder/bowel, sensory) FSS compared with last visit when the last |
| dysfunction | | | visit score ≥ 1 AND a new Gd-enhancing or new/enlarging T2 MRI |
| Lhermitte's sign (not | | | lesion in the spinal cord |
| in isolation) | | 15. | Worsening of ≥ 0.5 points in EDSS score compared with last visit if |
| | | | previous EDSS score ≥ 5.5 AND a new GD-enhancing or |
| | | | new/enlarging T2 MRI lesion in the spinal cord |
| Nausea | Brainstem | 16. | Isolated (not present at last visit) intractable nausea, vomiting, and/or |
| Intractable vomiting | | | hiccups lasting > 48 hours AND a new Gd-enhancing or new/enlarging |
| Intractable hiccups | | | T2 MRI lesion in the brainstem |
| Other neurological | | 17. | Worsening of ≥ 2 points in at least one of the relevant (brainstem, |
| signs (e.g. double | | | cerebellar) FSS compared with last visit AND a new Gd-enhancing or |
| vision, dysarthria, | | | new/enlarging T2 MRI lesion in the brainstem |
| dysphagia, vertigo, | | | |
| oculomotor palsy, | | | |
| weakness, | | | |
| nystagmus, other | | | |
| cranial nerve | | | |
| abnormality) | | | |
| Encephalopathy | Brain | 18. | Worsening of ≥ 2 points in at least one of the relevant (cerebral, |
| Hypothalamic | | | sensory, pyramidal) FSS (with a score of ≥ 3 at the current visit) |
| dysfunction | | | compared with last visit AND a new Gd-enhancing or new/enlarging |
| | | | T2 MRI lesion in the brain consistent with the clinical presentation |

*Symptoms listed are examples and are not inclusive of all neuromyelitis optica spectrum disorder symptoms.
†Four major areas of the body may be affected by an attack: the optic nerve, resulting in ON; the spinal cord, resulting in myelitis; the brainstem, resulting in several outcomes; and the brain.
‡A drop of ≥ 2 steps can be any of the following worsening: on Landolt C Broken Ring Chart to HM, LP, or NLP; CF to LP or NLP; HM to NLP.
§A drop of ≥ 1 steps can be any of the following worsening: on Landolt C Broken Ring Chart to CF, HM, LP, or NLP; CF to HM or LP or NLP; HM to LP or NLP; LP to NLP.
¶A 1-point change in a single FSS without a change in EDSS score, with or without a new Gd-enhancing or new/enlarging T2 MRI lesion in the spinal cord, is not considered a clinically significant change and does not count as an attack per this protocol.
∥Lesions seen in the optic chiasm also count towards these criteria.
CF, counting fingers;
EDSS, Expanded Disability Status Scale;
FSS, functional system score;
Gd, gadolinium;
HM, hand motion;
LP, light perception;
MRI, magnetic resonance imaging;
NLP, no light perception;
ON, optic neuritis;
RAPD, relative afferent pupillary defect.

Assessment Visits for subjects experiencing an NMO/NMOSD attack. Subjects who had experienced an NMO/NMOSD attack that met the protocol-defined criteria and required rescue treatment, regardless of the outcome of the Adjudication Committee review, would have undergone an Attack Follow-up Visit. Table 7 shows the procedures to have been conducted for the Attack Follow-up Visit. Patient reported outcomes should have be performed first followed by all other assessments/procedures in an order determined by the site. An attack follow-up visit should have been conducted 28 days from Day 1 of the Assessment Visit. This visit may have corresponded with an open-label period or safety follow-up visit, or may have been scheduled separately.

TABLE 7
Schedule of Study Procedures for the Attack Follow-Up Visit

| Procedure | Follow-up Assessment Visit^{a} |
|---|---|
| | 28 days (±7 days) After |
| Visit Window | Assessment Visit |
| Pain NRS | X |
| Vital signs | X |
| Modified Rankin Scale | X |
| SF-36 Health Survey (1-week recall version) | X |
| C-SSRS (“Since Last Visit” version)c | X |
| Physical/neurological examination | X |
| Collect blood for: | |
| Hematology and serum chemistry | X |
| Whole blood for flow cytometry (B-cell count | X |
| and cell subsets) | |
| Serum for AQP4-IgG assay (titers) | X |
| Collect urine for urinalysis | X |
| Independent ophthalmology examination | X^{b} |
| (high-ilow-contrast visual acuity test, RAPD | |
| assessment) | |
| Independent EDSS/FSS administration | X^{b,c} |
| Assessment of AEs and SAEs | X |
| Concomitant medications | X |

ADA = anti-drug antibody;
AE = adverse event;
AQP4-IgG = autoantibodies against aquaporin-4;
C-SSRS = Columbia Suicide Severity Rating Scale;
EDSS = Expanded Disability Status Scale;
FSS = Functional Systems Scores;
Ig A/E/G/M = immunoglobulin A/E/G/M;
NRS = numeric rating scale;
ON = optic neuritis;
RAPD = relative afferent pupillary defect;
SAE = serious adverse event:
SF-36 = Short Form-36 Health Survey.
^{a}The Follow-up Assessment Visit may be combined with an OLP or SFP visit if it occurs within the time window allowed for this visit.
^{b}Conduct the assessment(s) relevant to the type of attack only (eg, for ON attacks conduct the ophthalmology examination only).
^{c}If the EDSS and the C-SSRS are performed by the same person, the EDSS must be done first.

Safety Follow-Up Period. The safety follow-up period is started when a subject prematurely discontinues from the randomized control period or the open-label period. Procedures to be conducted during the safety follow-up period are provided in Table 8. The length of the safety follow-up period will have been determined by the time elapsed from the time of last dose of VIB551 to the time of the premature discontinuation, to complete a total of 52 weeks. Subjects who prematurely discontinued during the randomized control period will have been continuing with the study assessments until day 197, unless the consent has specifically been withdrawn for further study assessments.

TABLE 8
Schedule of Procedures for Safety Follow-Up Period

| Study Period | Follow-up Period |
|---|---|
| | Every 3 months for a total of |
| Visit Number | 1 year after last dose of |
| Procedure/Study | investigational product (±14d) |
| Vital signs | X |
| C-SSRS (“Since Last Visit” version) | X |
| Whole blood for flow cytometry | X |
| (B-cell count and cell subsets) | |
| Total Ig, IgM, IgG, IgA, and IgE | X |
| MEDI-551 ADA | X |
| Assessment of AEs/SAEs | X |

ADA = anti-drug antibody;
AE = adverse event;
C-SSRS = Columbia Suicide Severity Rating Scale;
d = day(s);
Ig A/E/G/M = immunoglobulin A/E/G/M;
SAE = serious adverse event

### Example 5—Attack Adjudication

Adjudication process for neuromyelitis optica spectrum disorder attacks. To ensure unified application of the defined attack diagnostic criteria, a detailed process of attack diagnosis and real-time adjudication was developed and followed. See Table 6. Patients were monitored for new or worsening symptoms related to neuromyelitis optica spectrum disorder attacks during scheduled study visits, and with follow-up phone calls every 2 weeks between study visits (or if a scheduled visit was missed).

When a possible new or worsening symptom(s) related to neuromyelitis optica spectrum disorder was identified, patients were required to inform the site. If an assessment visit was necessary, it was scheduled as soon as possible and within 72 hours of the report of the symptoms. At the assessment visit, patients were evaluated to determine if the symptoms were related to neuromyelitis optica spectrum disorder. If so, patients underwent further evaluations to determine if the symptoms met at least one of the protocol-defined criteria for a neuromyelitis optica spectrum disorder attack.

When a new or worsening symptom(s) did not meet at least one of the protocol-defined criteria for a neuromyelitis optica spectrum disorder attack, the patient continued in the randomised controlled period. Data related to the assessment of the symptoms that were determined by the investigator as not related to neuromyelitis optica spectrum disorder were sent to the adjudication committee for review.

Assessment of new symptoms or worsening of existing symptoms had to be completed within 5 days to determine if an attack had occurred. Treatment of an attack was initiated as appropriate after completion of the attack assessment and the determination that the protocol attack criteria had been met; however, the principal investigator was able to initiate rescue therapy at any time before if required. Rescue therapy was given as directed by the investigator. This may have included intravenous corticosteroids, intravenous immunoglobulin, and/or plasma exchange.

On completion of the assessment visit, the complete data set generated from the assessments was sent to the adjudication committee, regardless of whether a neuromyelitis optica spectrum disorder attack was diagnosed according to the protocol criteria by the principal investigator. The data sent included: (1) description of the new or worsening symptom; (2) findings of physical and neurological examinations; (3) relevant laboratory test results; (4) relevant X-ray studies, if performed in relation to the assessment; (5) Expanded Disability Status Scale (EDSS) score, as determined by the independent assessor; (6) ophthalmology examination results, conducted by an independent ophthalmologist; (7) MRI scans as required, depending on the suspected per-protocol attack criteria; (8) short narrative written by the principal investigator to summarise the assessment without disclosing if a neuromyelitis optica spectrum disorder attack was diagnosed (narrative template provided by the sponsor.

The adjudication committee was not provided with the principal investigator's opinion of whether a neuromyelitis optica spectrum disorder attack had occurred or not, nor was the adjudication committee sent information about which protocol attack criterion was met or whether or not a rescue medication was provided.

The adjudication process was completed within 14 days (+3 days) from initiation by the independent, blinded adjudication committee of three experts (discussed above). The adjudication committee determination was communicated to the principal investigator. Only adjudication-committee-determined attacks were used for the primary analysis. An adjudication decision was reached independently by each adjudicator and the outcome was based on majority vote.

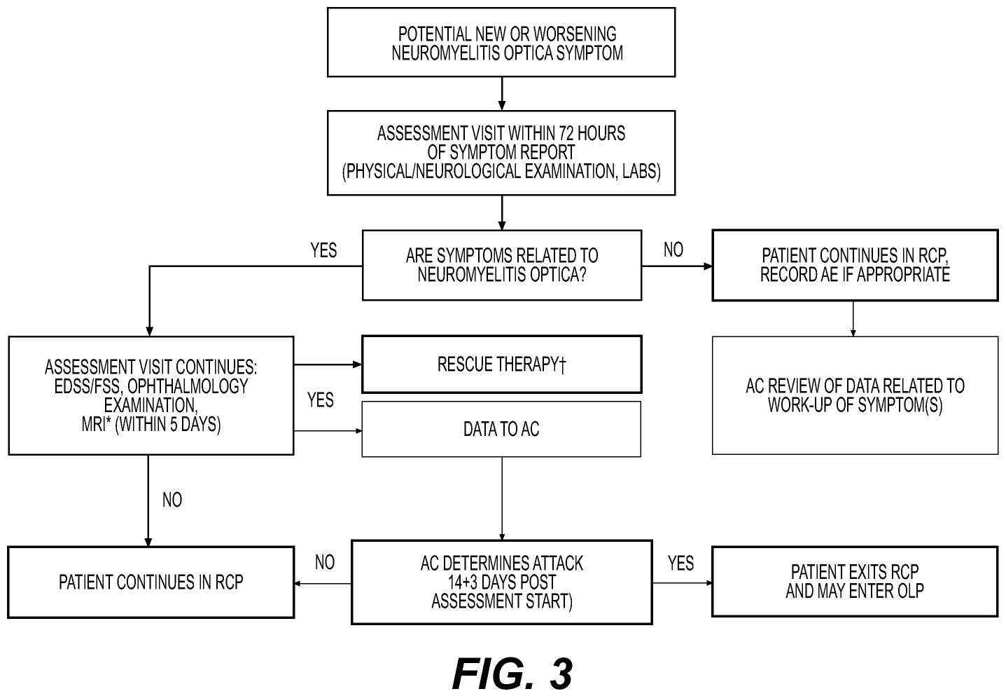

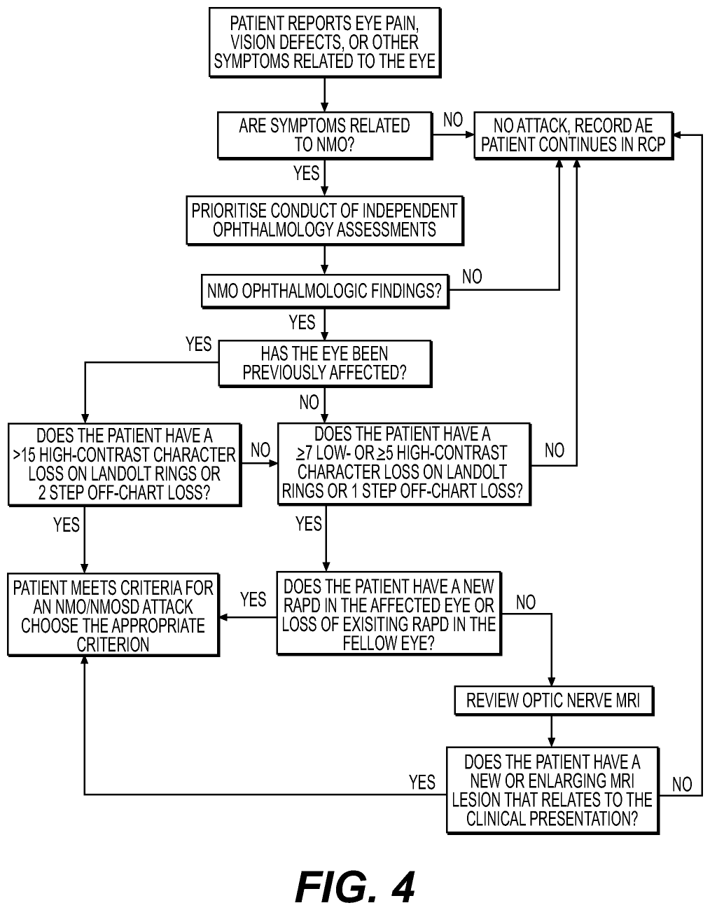

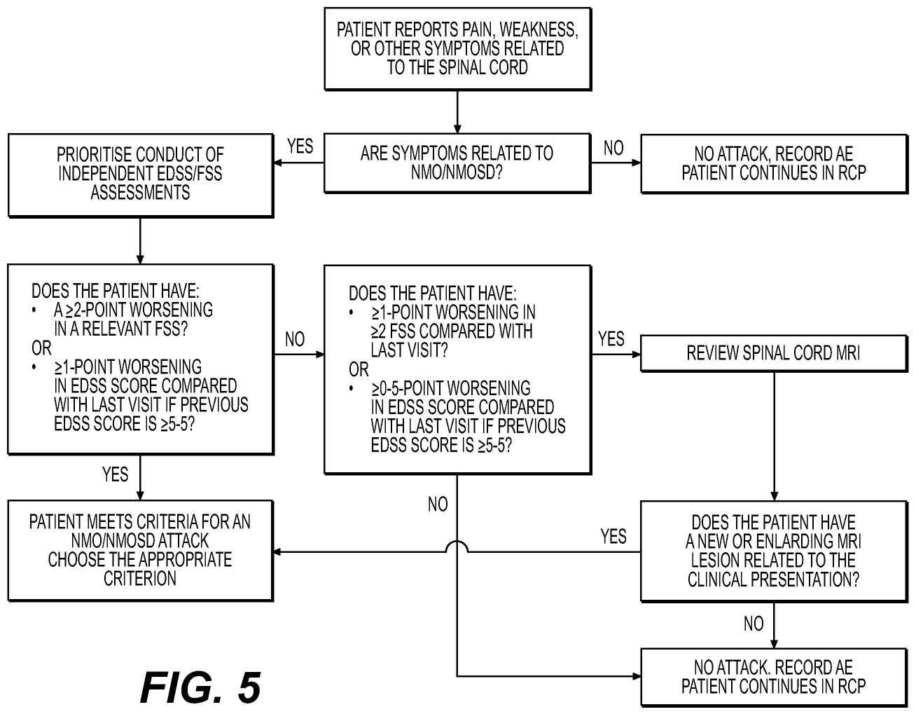

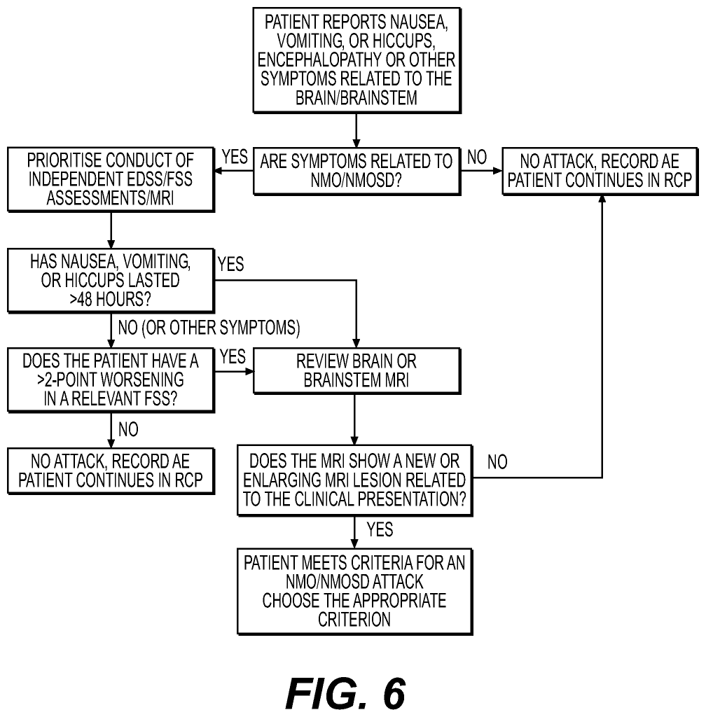

To minimise bias, both the Kurtzke EDSS rater and the ophthalmologist who conducted assessments used for adjudication of attacks were blinded to study treatment and other patient information, and were not part of the patient's treating team. To ensure the primacy of clinical findings in determination of attacks, the principal investigator was not allowed to review the MRI scans at the time of attack unless a specific attack diagnosis criterion required review of the relevant domain MRI. Flow diagrams indicating the process of assessing attacks, including for symptoms affecting the eye, spinal cord, or brain/brainstem, are included in FIGS. 3-6.

Patients for whom the diagnosis of a neuromyelitis optica spectrum disorder attack was not determined by the adjudication committee were given the option to continue in the randomised, controlled period until day 197. Patients for whom the diagnosis of a neuromyelitis optica spectrum disorder attack was determined by the adjudication committee were given the option to enter the open-label period.

In addition, patients who experienced a neuromyelitis optica spectrum disorder attack that required rescue treatment and met the protocol-defined criteria, regardless of the outcome of the adjudication committee review, underwent an attack follow-up visit 28 days from day 1 of the assessment visit. This visit could correspond with an open-label period or safety follow-up period visit or could be scheduled separately.

If patients did not wish to enter the open-label period or decided to leave the randomised controlled period at any point, they would continue to the safety follow-up period (unless consent was withdrawn).

### Example 6—Summary of Studied Outcomes

Primary endpoint. Time (in days) from day 1 to onset of an adjudication-committee-determined neuromyelitis optica spectrum disorder attack on or before day 197. The definition of an attack was the presence of a new symptom(s) or worsening of an existing symptom(s) related to neuromyelitis optica that met at least one of the protocol-defined criteria for a neuromyelitis optica spectrum disorder attack.

Secondary endpoints. Four key secondary endpoints were considered for study-wise type 1 error control: (1) Worsening from baseline in EDSS score at last visit during the randomised, controlled period. The EDSS assessment in the study was performed by an independent and blinded assessor at each site using an electronic data capture system, developed by the University of Basel and Neurostatus GmBH, which contained an internal algorithm providing feedback to the assessor on inconsistencies in the EDSS assessment; (2) Change from baseline in low-contrast visual acuity binocular score measured by low-contrast Landolt C Broken Ring Chart at last visit during the randomised controlled period; (3) Cumulative total number of active MRI lesions (new gadolinium-enhancing or new/enlarging T2 lesions) during the randomised controlled period; (4) Number of neuromyelitis optica-related inpatient hospitalisations, with inpatient hospitalisation defined as more than an overnight stay.

Remaining secondary endpoints. (1) Annualised attack rate (total number of adjudicated attacks, normalised by person-years) during any exposure to VIB551; (2) Treatment-emergent adverse events, including treatment-emergent serious adverse events; (3) Laboratory measurements, as well as their changes or shift from baseline over time; (4) Pharmacokinetic profile of VIB551; (5) Incidence of anti-drug antibodies directed against VIB551 for the duration of the study, both pre-dose and post-dose for each patient.

Exploratory endpoints. (1) Change from baseline in the 4-week recall 36-item Short-Form Health Survey, version 2, physical component score and mental component score at the last visit during the randomised, controlled period; (2) Change from baseline in pain numeric rating scale in five locations at the last visit during the randomised controlled period; (3) B-cell counts (total and subsets); (4) Change from baseline in plasma cell gene signature; (5) Serum AQP4-IgG titres.

### Example 7—Trial Participant Characteristics

From January 2015 to October 2018, 467 participants were screened at 99 participating sites in 24 countries. Of these, 231 were enrolled, with 175 randomised to VIB551 (AQP4-IgG seropositive, n=161) and 56 to placebo (AQP4-IgG seropositive, n=52; FIG. 1). On 7 Sep. 2018, the data-monitoring committee recommended halting enrolment, owing to clear demonstration of efficacy and conditional power in excess of 99%, before the targets of 252 participants/67 adjudicated attacks were met. The sponsor discontinued enrolment on Sep. 21, 2018 before database lock and while remaining blinded to treatment assignment.

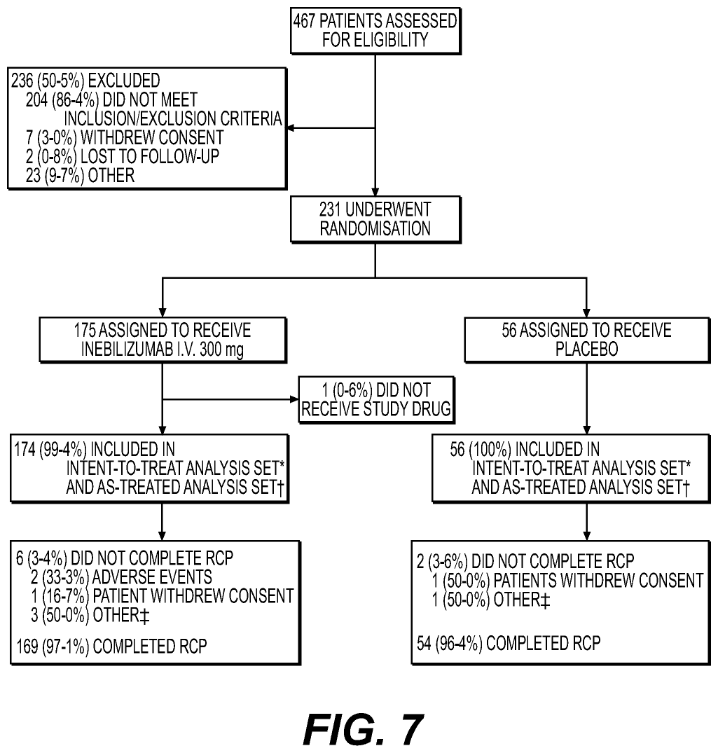

Of those assigned to VIB551, 174 (99.4%) were included in analysis populations (one participant [0.6%] did not receive study drug); 169 (97.1%) participants completed the randomised, controlled period, six discontinued owing to adverse events (n=2), withdrawn consent (n=1), or ‘other’ (n=3). All 56 participants assigned to placebo received intervention and were included in the analyses, with 54 (96.4%) completing the randomised, controlled period; two participants discontinued (n=1 withdrawn consent and n=1 ‘other’, FIG. 7). Most participants were women (n=209, 90.9%; table 9) and white (n=120, 52.2%, table 9). Participant demographics were broadly similar between treatment groups in the overall and AQP4-IgG seropositive populations (table 9). The open-label period is ongoing, with 213 participants receiving VIB551 (original randomisation: VIB551, n=162; placebo, n=51).

TABLE 9
Patient demographic and baseline characteristics

| | AQP4-IgG seropositive; n = 213 | | Overall ITT population; n = 230 | |
|---|---|---|---|---|
| | Placebo | VIB551 | Placebo | VIB551 |
| Demographic/characteristic | n = 52 | n = 161 | n = 56 | n = 174 |
| Age | | | | |
| Mean (SD), years | 42.4 (14.3) | 43.2 (11.6) | 42.6 (13.9) | 43.0 (11.6) |
| Sex | | | | |
| Women | 49 (94.2) | 151 (93.8) | 50 (89.3) | 159 (91.4) |
| Race | | | | |
| American Indian or Alaskan | 5 (9.6) | 11 (6.8) | 5 (8.9) | 14 (8.0) |
| Native | | | | |
| Asian | 8 (15.4) | 37 (23.0) | 8 (14.3) | 39 (22.4) |
| Black or African American | 5 (9.6) | 14 (8.7) | 5 (8.9) | 15 (8.6) |
| White | 24 (46.2) | 86 (53.4) | 28 (50.0) | 92 (52.9) |
| Other | 10 (19.2) | 12 (7.5) | 10 (17.9) | 13 (7.5) |
| Multiple categories checked | 0 | 1 (0.6) | 0 | 1 (0.6) |
| Ethnicity | | | | |
| Hispanic or Latino | 15 (28.8) | 25 (15.5) | 15 (26.8) | 28 (16.1) |
| Disease duration, years | | | | |
| Mean (SD) | 2.92 (3.54) | 2.49 (3.39) | 2.77 (3.45) | 2.41 (3.30) |
| Duration ≥5 years | 10 (19.2) | 29 (18.0) | 10 (17.9) | 30 (17.2) |
| Time since first relapse, years | | | | |
| Mean (SD) | 5.19 (5.69) | 5.19 (5.90) | 4.88 (5.59) | 5.12 (5.79) |
| Number of prior relapses | | | | |
| ≥2 relapses | 39 (75.0) | 137 (85.1) | 42 (75.0) | 149 (85.6) |
| Type of most recent attack | | | | |
| Optic neuritis | 19 (36.5) | 77 (47.8) | 21 (37.5) | 85 (48.9) |
| Myelitis | 32 (61.5) | 94 (58.4) | 34 (60.7) | 99 (56.9) |
| Brain/brainstem | 8 (15.4) | 6 (3.7) | 10 (17.9) | 8 (4.6) |
| Baseline AAR | | | | |
| Mean (SD) | 1.46 (1.36) | 1.68 (1.49) | 1.57 (1.46) | 1.73 (1.53) |
| Prior treatment | | | | |
| Any therapy* | 51 (98.1) | 159 (98.8) | 55 (98.2) | 172 (98.9) |
| Plasmapheresis | 26 (50.0) | 58 (36.0) | 27 (48.2) | 67 (38.5) |
| Intravenous immunoglobulin | 3 (5.8) | 8 (5.0) | 3 (5.4) | 8 (4.6) |
| Any prior maintenance therapy | 36 (69.2) | 108 (67.1) | 38 (67.9) | 114 (65.5) |
| Corticosteroids | 21 (40.4) | 74 (46.0) | 23 (41.1) | 79 (45.4) |
| Non-biologic immunosuppression^{†} | 25 (48.1) | 77 (47.8) | 26 (46.4) | 79 (45.4) |
| Azathioprine | 21 (40.4) | 62 (38.5) | 22 (39.3) | 63 (36.2) |
| Mycophenolate mofetil | 7 (13.5) | 25 (15.5) | 7 (12.5) | 26 (14.9) |
| Methotrexate | 0 | 2 (1.2) | 0 | 2 (1.1) |
| Biologic agent | 5 (9.6) | 23 (14.3) | 5 (8.9) | 25 (14.4) |
| Rituximab | 4 (7.7) | 12 (7.5) | 4 (7.1) | 13 (7.5) |
| Interferon beta | 1 (1.9) | 6 (3.7) | 1 (1.8) | 7 (4.0) |
| Natalizumab | 0 | 2 (1.2) | 0 | 2 (1.1) |
| Glatiramer acetate | 0 | 2 (1.2) | 0 | 2 (1.1) |
| No prior maintenance therapy | 16 (30.8) | 53 (32.9) | 18 (32.1) | 60 (34.5) |
| Baseline Gd-enhancing lesions | | | | |
| Mean (SD) | 0.8 (0.9) | 1.2 (1.2) | 0.9 (0.9) | 1.2 (1.2) |
| Median (range) | 1.0 (0.0-4.0) | 1.0 (0.0-5.0) | 1.0 (0.0-4.0) | 1.0 (0.0-5.0) |
| Baseline EDSS score | | | | |
| Mean (SD) | 4.35 (1.63) | 3.81 (1.77) | 4.19 (1.68) | 3.81 (1.81) |
| Median (range) | 4.0 (1.0-8.0) | 3.5 (0.0-8.0) | 4.0 (1.0-8.0) | 3.5 (0.0-8.0) |
| Weight, kg | n = 52 | n = 160 | n = 56 | n = 173 |
| Mean (SD) | 71.79 (19.97) | 68.16 (17.55) | 71.61 (19.26) | 68.37 (17.42) |
| Height, cm | n = 52 | n = 159 | n = 56 | n = 172 |
| Mean (SD) | 161.81 (7.25) | 163.86 (7.73) | 162.55 (7.73) | 164.30 (7.97) |
| BMI, kg/m² | n = 52 | n = 159 | n = 56 | n = 172 |
| Mean (SD) | 27.30 (6.90) | 25.29 (5.64) | 27.02 (6.73) | 25.22 (5.51) |
| BMI category | n = 52 | n = 159 | n = 56 | n = 172 |
| Underweight (<18.5 kg/m²) | 1 (1.9) | 10 (6.3) | 1 (1.8) | 10 (5.8) |
| Normal (18.5-<25.0 kg/m²) | 25 (48.1) | 79 (49.7) | 28 (50.0) | 85 (49.4) |
| Overweight (25.0-<30.0 kg/m²) | 10 (19.2) | 45 (28.3) | 11 (19.6) | 52 (30.2) |

### Example 8—VIB551 is Efficacious in Reducing Risk of an NMOSD Attack

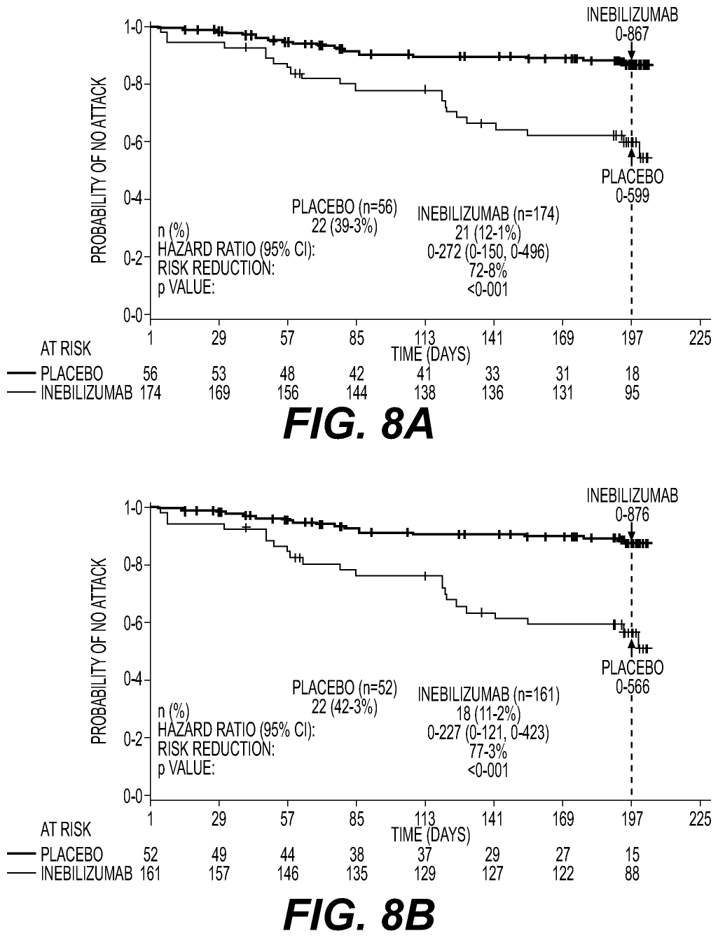

The study met the primary endpoint, with a significant difference in time to NMOSD attack in favour of VIB551 over placebo. Overall, 21/174 participants (12.1%) receiving VIB551 had an attack compared with 22/56 participants (39.3%) receiving placebo; relative risk reduction, 72.8%; hazard ratio 0.272 (95% confidence interval [CI], 0.150-0.496, p<0.0001); and number needed to treat 3.73 (95% CI, 3.06-5.66) (FIG. 8A). In the AQP4-IgG seropositive subgroup, relative risk reduction was 77.3%; hazard ratio 0.227 (95% CI, 0.121-0.423; p<0.0001); and number needed to treat 3.23 (95% CI, 2.72-4.54) (FIG. 8B). After 1 year on VIB551 treatment, 85% of patients were free of an NMOSD attack. In the combined RCP and OLP safety analyses, mean VIB551 treatment duration is 1.5 years (range 0.2-3.7).

A breakdown of attack type is presented in Table 10. Of the 43 AC-determined attacks, 27 were myelitis, 20 ON, and 2 brainstem attacks, with six affecting more than one domain. MRI-requiring criteria were deemed to have been met 16 times by the AC for the 43 events. Of the 22 attacks in the placebo group, 10 (45%) were graded as major and 12 (55%) as minor, compared to six of 21 (29%) major and 15 of 21 (71%) minor attacks in the VIB551-treated group.

TABLE 10
Breakdown of on-study adjudicated attacks

| | AQP4-IgG seropositive | | Overall ITT population | |
|---|---|---|---|---|
| | N = 213 | | N = 230 | |
| | Placebo | VIB551 | Placebo | VIB551 |
| Attack Outcome | n = 52 | n = 161 | n = 56 | n = 174 |
| AC-determined | 22 (42.3%) | 18 (11.2%) | 22 (39.3%) | 21 (12.1%) |
| attack* | | | | |
| Attacks according | | | | |
| to type† | | | | |
| Brainstem | 2 (9.1%) | 0 | 2 (9.1%) | 0 |
| Myelitis | 14 (63.6%) | 11 (61.1%) | 14 (63.6%) | 13 (61.9%) |
| Optic neuritis | 10 (45.5%) | 8 (44.4%) | 10 (45.5%) | 10 (47.6%) |
| Brainstem and | 1 (4.5%) | 0 | 1 (4.5%) | 0 |
| myelitis | | | | |
| Brainstem and | 1 (4.5%) | 0 | 1 (4.5%) | 0 |
| optic neuritis | | | | |
| Myelitis and optic | 2 (9.1%) | 1 (5.6%) | 2 (9.1%) | 2 (9.5%) |
| neuritis | | | | |

*Presented as n (%) of the number of patients in each treatment arm.
†Presented as n (%) of the total number of adjudicated attacks in each treatment arm. An attack may appear in more than one category.
AC, adjudication committee;
AQP4-IgG, aquaporin-4 immunoglobulin G;
ITT, intent-to-treat.

Recovery from attacks was graded as shown in Table 11. Recovery from attacks was assessed using an exploratory predefined scale (major, minor, no recovery) by the degree of domain-specific neurological recovery at the attack follow-up visit (within 35 days of the attack) compared to the attack visit. Among the 17 attacks with follow-up data in the placebo group, 9 (53%) exhibited no recovery and 8 (47%) had at least partial recovery. In the VIB551 group, 6/13 attacks (46%) exhibited no recovery and 7/13 (54%) had at least partial recovery.

TABLE 11
AC-determined NMOSD attacks by recovery grades, randomized
control period (intent-to-treat population)

| | AQP4-1gG sero+ | | | AQP4-IgG sero− | | Total | | | |
|---|---|---|---|---|---|---|---|---|---|
| | N = 213 | | | N = 17 | | N = 230 | | | |
| | Placebo | | Inebilizumab | Placebo | Inebilizumab | Placebo | | Inebilizumab | |
| | N = 52 | | N = 161 | N = 4 | N = 13 | N = 56 | | N = 174 | |
| AC-determined | 22 | (42.3%) | 18 (11.2%) | 0 | 3 (23.1%) | 22 | (39.3%) | 21 | (12.1%) |
| attack | | | | | | | | | |
| Attack | | | | | | | | | |
| recovery grade | | | | | | | | | |
| Major | 2 | (9.1%) | 2 (11.1%) | 0 | 0 | 2 | (9.1%) ^{a} | 2 | (9.5%) ^{b} |
| Minor | 6 | (27.3%) | 5 (27.8%) | 0 | 0 | 6 | (27.3%) ^{a} | 5 | (23.8%) ^{b} |
| No recovery | 9 | (40.9%) | 4 (22.1%) | 0 | 2 (66.7%) | 9 | (40.9%) ^{a} | 6 | (28.6%) ^{b} |

AC = Adjudication Committee; AQP4-IgG = autoantibodies against aquaporin-4; ITT = intent-to-treat; NMOSD = neuromyelitis optica spectrum disorders; ON = optic neuritis; RCP = randomized-controlled period; sero+ = seropositive; sero− = seronegative.
^{a} In the placebo group, the number of subjects for whom recovery data was collected was 17. Using this as a denominator to calculate percentages yields the following: Major, 11.8%; Minor, 35.5%; No recovery, 52.9%.
^{b} In the inebilizumab group, the number of subjects for whom recovery data was collected was 13. Using this as a denominator to calculate percentages yields the following: Major, 15.4%; Minor, 38.5%; No recovery, 46.2%.

An annualized attack rate (total number of AC-determined NMOSD attacks normalized by person years) during any exposure to VIB551 was also determined. Of note, an annualized attack rate could not also be calculated for the placebo treatment period because subjects were removed from the placebo-controlled portion of the study after an AC-adjudicated attack. Accordingly, any such calculation in the placebo period would have been biased and would have potentially overestimated the attack rate. Subjects in the VIB551 treatment group, however, remained in the study receiving VIB551 following an attack, and therefore, an estimate of annualized attack rate could be calculated for the period in which subjects were treated with VIB551.

The annualized AC-determined NMOSD attack rate in any subject treated with VIB551 was low, at 0.126. See Table 12. When calculated separately for the AQP4-IgG seropositive and AQP4-IgG seronegative subjects, the annualized attack rate was 0.13 and 0.088, respectively.

TABLE 12
Annualized Adjudication Committee-determined
NMOSD Attack Rate (Any VIB551 Population)

| | AQP4-IgG | AQP4-IgG | |
|---|---|---|---|
| | sero+ | sero− | Total |
| | N = 208 | N = 17 | N = 225 |
| Number of AC- | 42 | 3 | 45 |
| determined attack | | | |
| Total person-year ^{a} | 323.595 | 34.152 | 357.747 |
| Annualized attack | 013 | 0.088 | 0.126 |
| rate ^{b} | | | |

AC = Adjudication Committee;
AQP4-IgG = autoantibodies against aquaporin-4;
SFP = safety follow-up period;
sero− = seronegative;
sero+ = seropositive.
^{a} Total person-years will be calculated as the sum of the person-years for individual subject.
Person-year for individual subject is defined as (Date of last day before SFP − 1st inebilizumab dose date + 1)/365.25.
^{b} Annualized attack rate is defined as total number of AC-determined attacks divided by total person-years.

The study was stopped early on the recommendation of the independent data-monitoring committee, owing to the clear demonstration of efficacy.

### Example 9—VIB551 Met Key Secondary Endpoints of Reducing EDSS Score Worsening from Baseline, Reducing Active MRI Lesion Count and Reducing Disease-Related Inpatient Hospitalizations

Key secondary endpoints are presented for the overall intent-to-treat and AQP4-IgG seropositive populations in Table 13. Worsening in EDSS score from baseline to last visit of the RCP occurred in a lower proportion of subjects in the VIB551 group than the placebo group among AQP4-IgG seropositive subjects. This difference was statistically significant with odds ratio of 0.371 (95% CI: 0.1807, 0.7633; p=0.0070) for the VIB551 group compared to the placebo group. Similarly, in the overall ITT population, a lower proportion of subjects in the VIB551 total group than the placebo total group had a worsening in EDSS score. The odds ratio was 0.370 (95% CI: 0.1850, 0.7389; p=0.0049) for the VIB551 total group compared to the placebo total group. A subject was considered to have a worsening in overall EDSS score if one of the following criteria was met: worsening of 2 or more points in EDSS score for subjects with baseline score of 0; worsening of one or more points in EDSS score for subjects with baseline score of 1-5; worsening of 0.5 points or more in EDSS score for subjects with baseline score of 5.5 or more.

There was no difference in change from baseline in low-contrast visual acuity binocular score between treatment groups; however, VIB551-treated participants were less likely to experience optic neuritis; overall intent-to-treat population: hazard ratio 0.288 (95% CI, 0.120-0.694); AQP4-IgG seropositive population: hazard ratio 0.222 (95% CI, 0.088-0.565).

Treatment with VIB551 significantly reduced the cumulative number of active MRI lesions compared to treatment with placebo. The rate ratio between the groups was 0.568 (95% CI: 0.3851, 0.8363; p-0.0042) among AQP4-IgG seropositive subjects (Table 13). For subjects with active MRI lesions, the mean cumulative number of active MRI lesions was lower in the VIB551 group (1.7 lesions) compared to the placebo group (2.3 lesions) among AQP4-IgG seropositive subjects. In the overall ITT population, similar results were observed. It should be noted that the mean number of lesions is calculated based on all subjects in each population.

Treatment with VIB551 significantly reduced the number of in-patient hospitalizations compared to treatment with placebo. The rate ratio between the groups was 0.258 (95% CI: 0.0904, 0.7384; p=0.0115) among AQP4-IgG seropositive subjects (Table 13). For subjects with hospitalizations, the mean number of NMOSD-related in-patient hospitalizations was lower in the inebilizumab group than the placebo group among AQP4-=IgG seropositive subjects. In the overall ITT population, a similar significant difference was observed.

TABLE 13
Key secondary outcomes

| | AQP4-IgG | | | Overall ITT | | |
|---|---|---|---|---|---|---|
| | seropositive; n = 213 | | | population; n = 230 | | |
| | Placebo | VIB551 | | Placebo | VIB551 | |
| Secondary endpoint | n = 52 | n = 161 | p value* | n = 56 | n = 174 | p value* |
| Worsening from baseline in | n = 52 | n = 161 | | n = 56 | n = 174 | |
| EDSS score at last visit^{†} | | | | | | |
| n (%) | 18 (34.6) | 25 (15.5) | | 19 (33.9) | 27 (15.5) | |
| OR (95% CI) | 0.371 (0.181-0.763) | | 0.021 | 0.370 (0.185-0.739) | | 0.021 |
| Change from baseline in | n = 52 | n = 158 | | n = 56 | n = 171 | |
| LCVAB score^{‡} | | | | | | |
| LSM (SE) | 0.600 (0.999) | 0.562 (0.572) | | 1.442 (1.217) | 1.576 (0.935) | |
| LSM difference (95% CI) | −0.038 (−2.312-2.236) | | 0.974 | 0.134 (−2.025-2.294) | | 0.974 |
| Cumulative number of active | n = 31 | n = 74 | | n = 32 | n = 79 | |
| MRI lesions^{§} | | | | | | |
| Mean (SD) | 2.3 (1.3) | 1.7 (1.0) | | 2.3 (1.3) | 1.6 (1.0) | |
| RR (95% CI)^{¶} | 0.568 (0.385-0.836) | | 0.017 | 0.566 (0.387-0.828) | | 0.017 |
| Cumulative number of | n = 7 | n = 8 | | n = 8 | n = 10 | |
| inpatient hospitalisations^{∥} | | | | | | |
| Mean (SD) | 1.4 (0.8) | 1.0 (0.0) | | 1.4 (0.7) | 1.0 (0.0) | |

| | | | | |
|---|---|---|---|---|
| RR (95% CI)^{¶} | 0.258 (0.090-0.738) | 0.23 | 0.286 (0.111-0.741) | 0.023 |

*Presented p values are adjusted for multiple comparison testing; differences were considered significant if p < 0.05.
^{†}Proportion of participants with worsening EDSS score from baseline, with OR calculated using a logistic regression model with treatment, serostatus, and baseline score as explanatory variables and non-responder imputation (with missing values considered as worsening).
^{‡}LSM differences in the change in LCVAB score were assessed using an analysis of covariance model with treatment, serostatus, and baseline Landolt C Broken Ring Chart binocular score as explanatory variables and last non-missing low-contrast visual acuity score.
^{§}Cumulative number of active MRI lesions from baseline (includes gadolinium-enhancing or new/enlarging T2 lesions), with RRs assessed using negative binomial regression, with treatment and serostatus as explanatory variables
^{¶}RR analysis is based on the entire population, not just those who had an event.
^{∥}Cumulative number of neuro myelitis optica-related inpatient hospitalisations from baseline, with RRs assessed using negative binomial regression, with treatment and serostatus as explanatory variables.
AQP4-IgG, 161 aquaporin-4-immunoglobulin G; CI, confidence interval; EDSS, Expanded Disability Status Scale; ITT, intent-to-treat; LCVAB, low-contrast visual acuity binocular; LSM, least-squares mean; MRI, magnetic resonance imaging; OR, odds ratio; RR, rate ratio; SD, standard deviation; SE, standard error.

### Example 10—Changes in Baseline in the mRS for Neurological Disability Indicated a Significant Benefit for VIB551-Treated NMOSD Subjects

Treatment effect based on mRS score during the RCP was evaluated by the Wilcoxon-Mann-Whitney odds approach. The mRS is a commonly used scale for measuring the degree of disability or dependence in the daily activities of people who have suffered a stroke or other causes of neurological disability. The mRS ranges from 0-6, ranging from perfect health without symptoms to death, as follows: 0—no symptoms; 1—no significant disability, able to carry out all usual activities, despite some symptoms; 2—slight disability, able to look after own affairs without assistance, but unable to carry out all previous activities; 3—moderate disability, requires some help, but able to walk unassisted; 4—moderately severe disability, unable to attend to own bodily needs without assistance, and unable to walk unassisted; 5—severe disability, requires constant nursing care and attention, bedridden, incontinent; 6—dead.

AQP4-IgG seropositive subjects who had received VIB551 were 74.2% more likely to report less disability compared to placebo subjects. In 52.8% of possible pairs of VIB551 and placebo subjects, the VIB551 treated subjects had a better outcome than the placebo subjects at the last visit. In 25.7% of pairs, placebo subjects had a better outcome and in 21.5% of pairs, VIB551 subjects were tied with placebo subjects. This lead to Wilcoxon-Mann-Whitney odds of 1.742 (p=0.0014).

In the overall ITT population, in 51.5% of pairs, VIB551 subjects had a better outcome than placebo subjects; in 26.6% of pairs, placebo subjects had a better outcome; and in 21.9% of pairs mRS was tied. Subjects who received VIB551 were 66.3% more likely to report less disability compared to placebo subjects.

After a period of only six and a half months, the VIB551-treated subjects had a reduced risk of worsening disability as measured by mRS. The VIB551-treated subjects not only had a reduced risk of worsening disability as measured by mRS, they also had a reduced risk of worsening disability as measured by EDSS.

### Example 11—VIB551-Treated NMOSD Subjects Experienced Less Extremities Pain

Overall, the mean change from baseline to week 28 in average pain NRS score for all body locations was similar in the placebo group and the VIB551 group. However, among the AQP4-IgG seropositive subjects and in the total intent-to-treat population, there was a trend showing a smaller mean increase in leg pain in the VIB551 group than in the placebo group. See Table 14.

TABLE 14
Change from baseline in pain NRS at the last visit using analysis of covariance model, randomized-controlled period (intent-to-treat population)

| | | AQP4-IgG sero+ | | | AQP4-IgG sero− | | | Total | | |
|---|---|---|---|---|---|---|---|---|---|---|
| | | N = 213 | | | N = 17 | | | N = 230 | | |
| | | Placebo | Inebilizumab | | Placebo | Inebilizumab | | Placebo | Inebilizumab | |
| Endpoints | | N = 52 | N = 161 | | N = 4 | N = 13 | | N = 56 | N = 174 | |
| Average pain | n | 51 | 159 | | 4 | 13 | | 55 | 172 | |
| score over | | | | | | | | | | |
| all body | | | | | | | | | | |
| locations^{a} | | | | | | | | | | |
| | Observed mean | 0.514 (0.304) | 0.299 | (0.118) | 1.550 (0.624) | 0.754 | (0.387) | 0.589 (0.287) | 0.334 | (0.113) |
| | (SE) | | | | | | | | | |
| | LS mean (SE)^{b} | 0.567 (0.229) | 0.282 | (0.130) | 1.592 (0.710) | 0.741 | (0.391) | 0.824 (0.278) | 0.496 | (0.213) |
| | LS mean | | −0.285 | (0.263) | | −0.852 | (0.814) | | −0.327 | (0.251) |
| | difference (SE)^{b} | | | | | | | | | |
| | 95% CI^{b} | | (−0.8038, 0.2348) | | | (−2.5974, 0.8941) | | | (−0.8213, 0.1664) | |
| | p-value^{b} | | 0.2813 | | | 0.3132 | | | 0.1927 | |
| Pain scores | n | 51 | 159 | | 4 | 13 | | 55 | 172 | |
| for eyes | | | | | | | | | | |
| | Observed mean | 0.667 (0.432) | 0.377 | (0.139) | 2.250 (1.109) | 0.308 | (0.499) | 0.782 (0.410) | 0.372 | (0.133) |
| | (SE) | | | | | | | | | |
| | LS mean (SE)^{b} | 0.807 (0.282) | 0.332 | (0.159) | 2.292 (0.625) | 0.295 | (0.347) | 1.076 (0.342) | 0.481 | (0.259) |
| | LS mean | | −0.475 | (0.325) | | −1.997 | (0.715) | | −0.596 | (0.307) |
| | difference (SE)^{b} | | | | | | | | | |
| | 95% CI^{b} | | (−1.1151, 0.1655) | | | (−3.5303, −0.4632) | | | (−1.2008, 0.0096) | |
| | p-value^{b} | | 0.1453 | | | 0.0144 | | | 0.0537 | |
| Pain scores | n | 51 | 159 | | 4 | 13 | | 55 | 172 | |
| for legs | | | | | | | | | | |
| | Observed mean | 1.078 (0.404) | 0.283 | (0.178) | 1.500 (0.645) | 0.615 | (0.730) | 1.109 (0.377) | 0.308 | (0.173) |
| | (SE) | | | | | | | | | |
| | LS mean (SE)^{b} | 1.182 (0.318) | 0.250 | (0.180) | 1.609 (1.209) | 0.582 | (0.669) | 1.297 (0.391) | 0.357 | (0.299) |
| | LS mean | | −0.932 | (0.366) | | −1.027 | (1.384) | | −0.939 | (0.352) |
| | difference (SE)^{b} | | | | | | | | | |
| | 95% CI^{b} | | (−1.6532-0.2104) | | | (−3.9953, 1.9415) | | | (−1.6333, −0.2453) | |
| | p-value^{b} | | 0.0116 | | | 0.4704 | | | 0.0082 | |
| Pain scores | n | 51 | 159 | | 4 | 13 | | 55 | 172 | |
| for arms | | | | | | | | | | |
| | Observed mean (SE) | 0.667 (0.408) | 0.377 | (0.190) | 0.000 (0.408) | 0.538 | (0.351) | 0.618 (0.380) | 0.390 | (0.178) |
| | LS mean (SE)^{b} | 0.627 (0.321) | 0.390 | (0.182) | 0.221 (8.721) | 0.471 | (0.358) | 0.374 (0.387) | 0.165 | (0.295) |
| | LS mean | | −0.237 | (0.369) | | 0.250 | (0.861) | | −0.209 | (0.346) |
| | difference (SE)^{b} | | | | | | | | | |
| | 95% CI^{b} | | (−0.9653, 0.4913) | | | (−1.5964, 2.0964) | | | (−0.8903, 0.4728) | |
| | p-value^{b} | | 0.5218 | | | 0.7758 | | | 0.5467 | |
| Pain scores | n | 51 | 159 | | 4 | 13 | | 55 | 172 | |
| for | | | | | | | | | | |
| upper back | | | | | | | | | | |
| | Observed mean | 0.039 (0.479) | 0.176 | (0.186) | 2.250 (1.315) | 1.000 | (0.776) | 6.200 (0.459) | 0.238 | (0.182) |
| | (SE) | | | | | | | | | |
| | LS mean (SE)^{b} | 0.111 (0.344) | 0.153 | (0.195) | 2.105 (1.281) | 1.045 | (8.710) | 0.532 (0.426) | 0.483 | (0.326) |
| | LS mean | | 0.042 | (0.395) | | −1.060 | (1.465) | | −0.050 | (0.381) |
| | difference (SE)^{b} | | | | | | | | | |
| | 95% CI^{b} | | (−0.7366, 0.8211) | | | (−4.2027, 2.0830) | | | (−0.8017, 0.7019) | |
| | p-value^{b} | | 0.9149 | | | 0.4814 | | | 0.8961 | |
| Pain, score | n | 51 | 159 | | 4 | 13 | | 55 | 172 | |
| for low | | | | | | | | | | |
| back | | | | | | | | | | |
| | Observed mean | 0.118 (0.451) | 0.270 | (0.215) | 1.750 (0.629) | 1.308 | (0.728) | 0.236 (0.424) | 0.349 | (0.207) |
| | (SE) | | | | | | | | | |
| | LS mean (SE)^{b} | 0.333 (0.352) | 0.201 | (0.199) | 1.752 (1.253) | 1.307 | (0.694) | 0.815 (0.433) | 0.658 | (0.330) |
| | LS mean | | −0.132 | (0.404) | | −0.445 | (1.435) | | −0.157 | (0.390) |
| | difference (SE)^{b} | | | | | | | | | |

| | | | |
|---|---|---|---|
| 95% CI^{b} | (−0.9288, 0.6656) | (−3.5216, 2.6320) | (−0.9253, −0.6116) |
| p-value^{b} | 0.7452 | 0.7611 | 0.6879 |

AQP4-IgG = autoantibodies against aquaporin-4; CI = confidence interval: LS = least squares: ITT = intent-to-treat; NRS = numeric rating score; SE = standard error; sero− = seronegative; sero+ = seropositive.
^{a}Average pain score over all body locations will be calculated over body locations with non-missing scores,
^{b}LS mean, LS mean difference, its 95% CI. and p-value are estimated by using an analysis of covariance model, using last non-missing result for each endpoint

It was also noted that pain in extremity was less common in the VIB551 group than the placebo group (0.6% versus 7.1% during the randomized controlled period; 4.9% in the any VIB551 population). This difference may reflect lower NMOSD activity, and is consistent with the finding of better outcome for leg pain as measured by the NRS, shown in Table 14 and discussed above.

### Example 12—VIB551 Depletes CD20+ B Cells and Decreases Immunoglobulin Levels of NMOSD Subjects

Peripheral blood mononuclear cell subsets including B cells (CD20+, transitional, naïve, memory B cells, plasmablasts and plasma cells), T cells (CD4+ and CD8+), and NK cells were assayed by flow cytometry (FACS) through the 28-week randomized control period (RCP). Peripheral blood B- and plasma cell-specific gene expression signatures were also assessed by reverse-transcriptase qPCR (rt-qPCR).

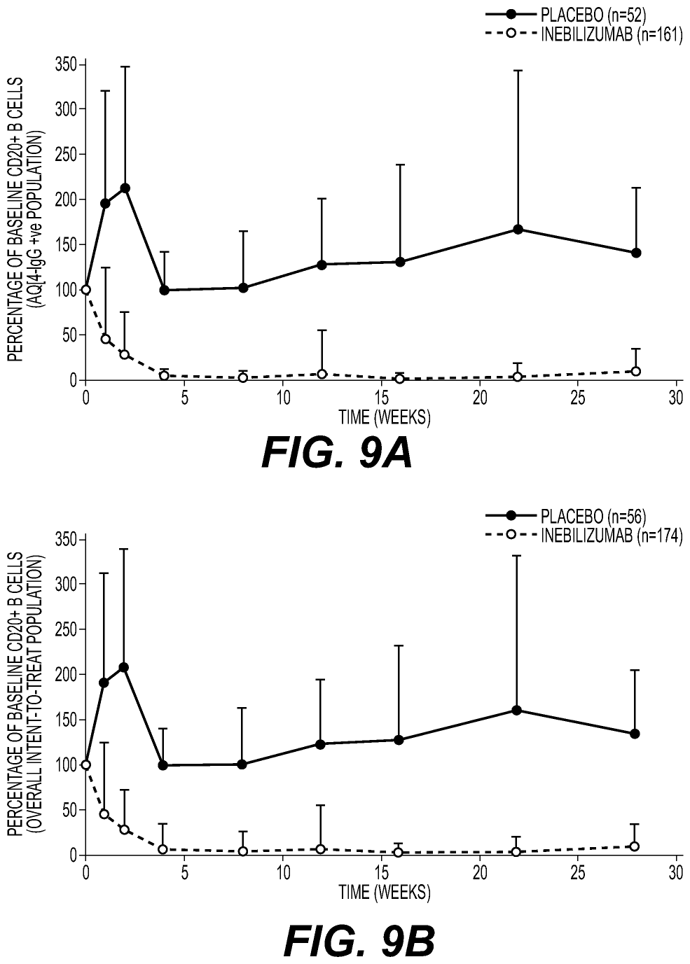

For both AQP4-IgG seropopulations, the pharmacodynamic effect of VIB551 on CD20+ B cells was observed within 4 weeks, with a significant and robust depletion of circulating B cells compared with placebo. B-cell counts in the VIB551 group dropped below 10% of baseline and remained under this threshold throughout the randomised, controlled period. See FIGS. 9A and 9B. No such effect was observed with placebo.

The CD20+ B-cell counts were significantly reduced and remained below the lower limit of normal (LLN; 74.4 CD20+ B-cells/uL) in 94% of patients for the randomized control period. T cell counts were unchanged; NK cells showed a transient decrease at day 15. Peripheral blood CD20− plasmablasts and plasma cells were rapidly depleted by day 8 and remained depleted throughout the randomized controlled period.

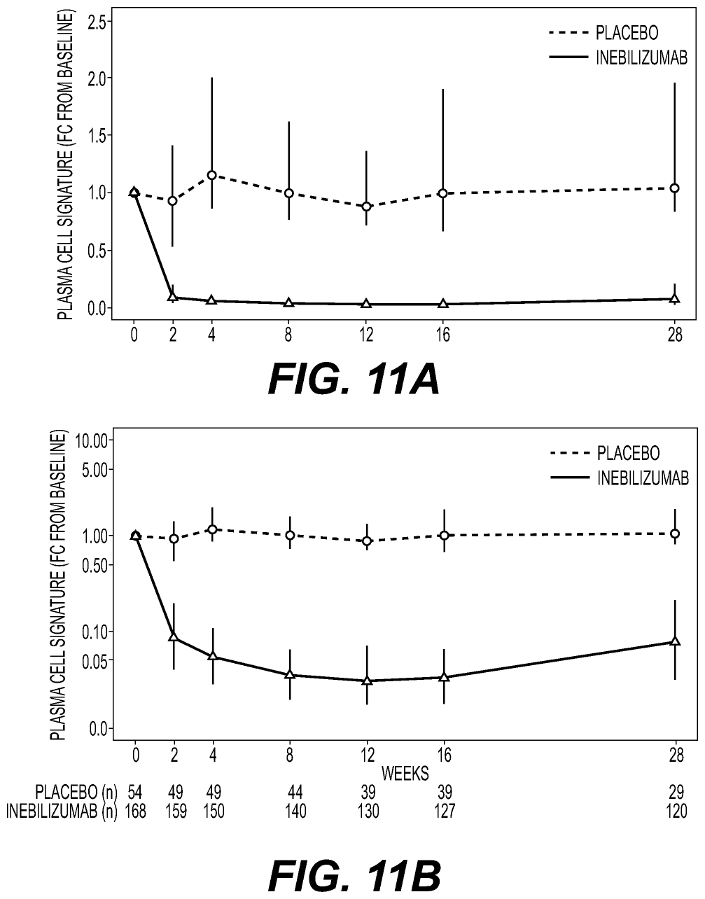

Plasma cell depletion was verified by plasma cell gene expression signature analysis. The plasma cell signature was based on expression analysis of four genes (IGHA1, IGJ, IGKV4-1, and TNFRSF17) expressed predominantly in plasma cells in blood. Plasma cell-specific gene expression signature was evaluated by reverse transcriptase quantitative PCR (rt-qPCR) of blood cell RNA samples collected on Day 1, 15, 29, 85, 113, 155, and 197 of the randomized controlled period. The signature was calculated as the average expression of the four plasma cell-specific genes minus the average expression of five control genes (B2M, GAPDH, TFRC, GUSB and UBC) at each time point. The fold-change in plasma cell gene expression signature was calculated at each time point relative to day 1 predose levels and was interpreted as a change in plasma cell abundance. The plasma cell-specific gene expression signature was significantly reduced in the VIB551-treated subjects by day 15, and it was decreased >10-fold during the 28 week randomized control period. The plasma cell-specific gene signature was not significantly different between any time points in the placebo group. See FIGS. 11A and 11B.

Subjects with complete FACS data were stratified by the depth and duration of B cell depletion. “Stable depleters [SD]” (74% of treated subjects) had B cell counts continuously <5 cells/uL during the RCP. “Partial depleters [PD]” (9% of treated subjects) had B cell counts <LLN but >5 cells/ul throughout the RCP. “Early reconstituters [ER]” (17% of treated subjects) showed initial B cell depletion but repleted to >5 cells/uL prior to the final RCP visit. Early repletion of B cells was due to the emergence of transitional B cells and naïve B cells. There was no relationship between the frequency of NMOSD attacks and CD20+ B-cell depletion (SD: 12/118 (10%), PD: 2/15 (13%), ER: 4/28 (14%)) in VIB551 treated subjects. Plasma cell gene signatures were not significantly higher in relapsing VIB551 treated subjects.

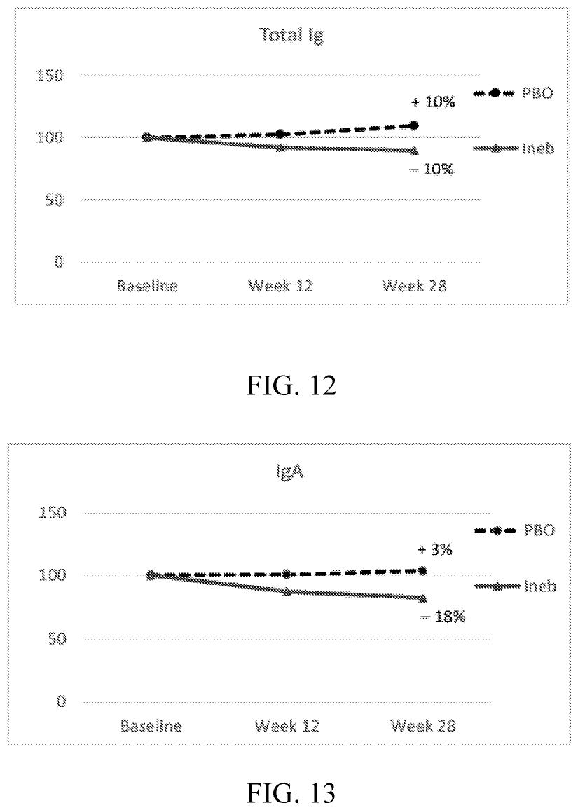

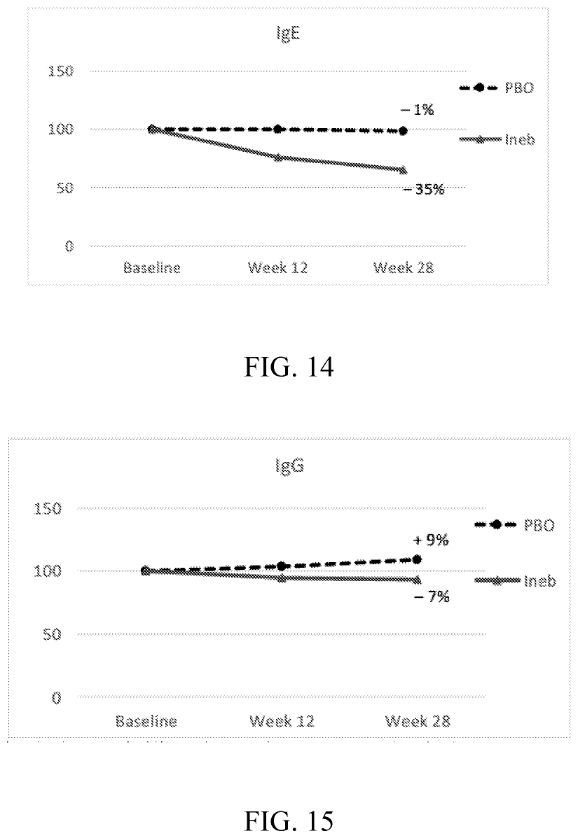

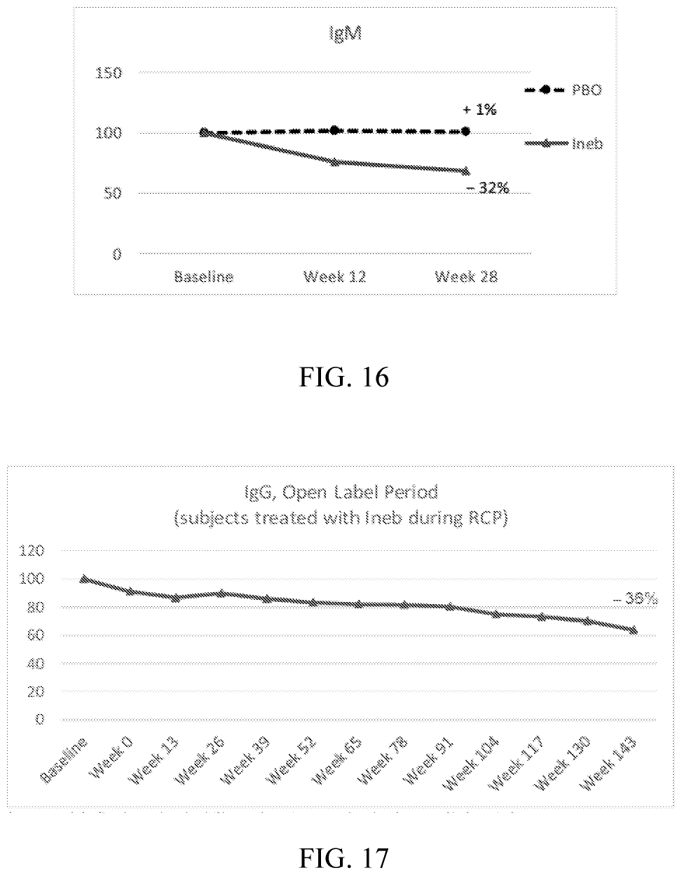

Consistent with known class effects of B cell depleting drugs, serum immunoglobulin levels also decreased with VIB551 use. In the subjects receiving VIB551 from the start of the randomized control period, the median percent change in total Ig levels at open label period week 0 was −12.42%, at open label period week 52 was −19.48%, at open label period week 104 was −28.59%, and at open label period week 143 was −40.12%. In the subjects receiving VIB551 from the start of the RCP, the median percent change in IgG level at open label period week 0 was −8.88%, at open label period week 52 was −16.69%, at open label period week 104 was −25.33% and at open label period week 143 was −36.12%. See FIGS. 12-17.

### Example 13—VIB551 is Safe for Treatment of NMOSD

Adverse events occurred in 71.8% of participants receiving VIB551 and 73.2% of those receiving placebo (Table 15). Most adverse events were more common with placebo than with VIB551, including infusion-related reaction (placebo, 10.7%; VIB551, 9.2%). Urinary tract infection, arthralgia, back pain, headache, fall, hypoaesthesia, cystitis, and eye pain were nominally more frequent with VIB551. Serious adverse events occurred in 4.6% of participants receiving VIB551 and 8.9% receiving placebo; none was reported in more than one participant, and no deaths occurred throughout the randomised, controlled period. Safety endpoints in the AQP4-IgG seropositive population were similar to those reported above.

TABLE 15
TEAEs and SAEs (as-treated population)

| | AQP4-IgG seropositive; | | Overall ITT population; | |
|---|---|---|---|---|
| | n = 213 | | n = 230 | |
| | Placebo | VIB551 | Placebo | VIB551 |
| Event* | n = 52 | n = 161 | n = 56 | n = 174 |
| Any AE | 37 (71.2) | 117 (72.7) | 41 (73.2) | 125 (71.8) |
| Any SAE | 5 (9.6) | 6 (3.7) | 5 (8.9) | 8 (4.6) |
| Any AE leading to dose interruption | 0 | 3 (1.9) | 0 | 3 (1.7) |
| Any AE leading to treatment discontinuation | 0 | 2 (1.2) | 0 | 2 (1.1) |

Most frequent AEs†

| | | | | |
|---|---|---|---|---|
| Urinary tract infection | 5 (9.6) | 18 (11.2) | 5 (8.9) | 20 (11.5) |
| Arthralgia | 2 (3.8) | 16 (9.9) | 2 (3.6) | 17 (9.8) |
| Infusion-related reaction | 5 (9.6) | 15 (9.3) | 6 (10.7) | 16 (9.2) |
| Back pain | 2 (3.8) | 11 (6.8) | 2 (3.6) | 13 (7.5) |
| Headache | 4 (7.7) | 13 (8.1) | 4 (7.1) | 13 (7.5) |
| Nasopharyngitis | 6 (11.5) | 12 (7.5) | 6 (10.7) | 13 (7.5) |
| Diarrhoea | 3 (5.8) | 7 (4.3) | 3 (5.4) | 8 (4.6) |
| Fall | 1 (1.9) | 7 (4.3) | 1 (1.8) | 8 (4.6) |
| Anaemia | 2 (3.8) | 6 (3.7) | 2 (3.6) | 6 (3.4) |
| Hypoaesthesia | 1 (1.9) | 5 (3.1) | 1 (1.8) | 6 (3.4) |
| Nausea | 2 (3.8) | 6 (3.7) | 3 (5.4) | 6 (3.4) |
| Cystitis | 0 | 4 (2.5) | 0 | 5 (2.9) |
| Eye pain | 1 (1.9) | 5 (3.1) | 1 (1.8) | 5 (2.9) |
| Upper respiratory tract infection | 3 (5.8) | 5 (3.1) | 3 (5.4) | 5 (2.9) |
| Depression | 5 (9.6) | 4 (2.5) | 5 (8.9) | 4 (2.3) |
| Influenza | 2 (3.8) | 4 (2.5) | 2 (3.6) | 4 (2.3) |
| Bronchitis | 2 (3.8) | 3 (1.9) | 2 (3.6) | 3 (1.7) |
| Hypertension | 2 (3.8) | 3 (1.9) | 2 (3.6) | 3 (1.7) |
| Influenza-like illness | 2 (3.8) | 3 (1.9) | 2 (3.6) | 3 (1.7) |
| Pyrexia | 1 (1.9) | 2 (1.2) | 2 (3.6) | 2 (1.1) |
| Alopecia | 2 (3.8) | 1 (0.6) | 2 (3.6) | 1 (0.6) |
| Myalgia | 2 (3.8) | 1 (0.6) | 2 (3.6) | 1 (0.6) |
| Oral herpes | 3 (5.8) | 1 (0.6) | 3 (5.4) | 1 (0.6) |
| Pain in extremity | 4 (7.7) | 1 (0.6) | 4 (7.1) | 1 (0.6) |
| Pruritus | 5 (9.6) | 1 (0.6) | 5 (8.9) | 1 (0.6) |
| Vomiting | 4 (7.7) | 1 (0.6) | 4 (7.1) | 1 (0.6) |
| Constipation | 2 (3.8) | 0 | 2 (3.6) | 0 |

SAEs‡§

| | | | | |
|---|---|---|---|---|
| Arthralgia | 0 | 1 (0.6) | 0 | 1 (0.6) |
| Atypical pneumonia | 0 | 1 (0.6) | 0 | 1 (0.6) |
| Burns third degree | 0 | 1 (0.6) | 0 | 1 (0.6) |
| Chest pain | 1 (1.9) | 0 | 1 (1.8) | 0 |
| Cholangitis acute | 0 | 1 (0.6) | 0 | 1 (0.6) |
| Cholecystitis acute | 0 | 1 (0.6) | 0 | 1 (0.6) |
| Diarrhoea | 0 | 1 (0.6) | 0 | 1 (0.6) |
| Dyspnoea | 1 (1.9) | 0 | 1 (1.8) | 0 |
| Hepatic function abnormal | 0 | 1 (0.6) | 0 | 1 (0.6) |
| Hypoglycaemia | 1 (1.9) | 0 | 1 (1.8) | 0 |
| Meningitis viral | 1 (1.9) | 0 | 1 (1.8) | 0 |
| Migraine | 1 (1.9) | 0 | 1 (1.8) | 0 |
| Myelitis | 0 | 1 (0.6) | 0 | 1 (0.6) |
| Pneumonia | 1 (1.9) | 0 | 1 (1.8) | 0 |
| Septic shock | 1 (1.9) | 0 | 1 (1.8) | 0 |
| Urinary tract infection | 0 | 0 | 0 | 1 (0.6) |
| Visual acuity reduced | 1 (1.9) | 0 | 1 (1.8) | 0 |
| Vision blurred | 0 | 0 | 0 | 1 (0.6) |

Data are n (%).
*All AEs and SAEs were coded using Medical Dictionary for Regulatory Activities version 21.0 and were reported descriptively.
Participants were counted once for each preferred term regardless of the number of events.
†Most frequent AEs included AEs experienced by > 2.5% of patients in either group of the overall ITT population.
‡No SAE was experienced by more than one patient in either group.
§Although no deaths occurred in the randomised-controlled period, two deaths were reported in the open-label period.
AE, adverse event;
AQP4-IgG, aquaporin-4 immunoglobulin G;
ITT, intent-to-treat;
SAE, serious adverse event;
TEAE, treatment-emergent adverse event

B-cell-depleting therapies have been associated with increased risk of cancer and infection, including PML.^{20,23} With VIB551, no malignancies occurred, and the infection rate was lower than that with placebo. Although no deaths occurred in the randomised, controlled period, two deaths were reported in the open-label phase. The first was a participant that had a serious adverse event of pneumonia shortly after randomisation, followed by an adjudicated attack, prior to enrolment into the open-label period. The participant died at home nine days later, probably from respiratory insufficiency. The second participant had new-onset weakness, aphasia, neurological decline, and seizures during the open-label period. Brain MRI demonstrated large new lesions involving both white- and grey-matter structures. The patient suffered respiratory arrest and died of the cardiopulmonary complications. Brain biopsy or autopsy was not done. Polymerase chain reaction testing of cerebrospinal fluid for John Cunningham virus (JCV) tested negative in two certified independent laboratories but was reported as positive in another unidentified laboratory. Without conclusive MRI or JCV information, the differential diagnosis included progressive multifocal leukoencephalopathy (PML), acute disseminated encephalomyelitis, and atypical NMOSD attack.

### Example 14—Subclinical MRI Lesion Activity is a Characteristic of NMOSD Disease

As a part of the clinical trial, longitudinal MRIs of patients with NMOSD were systematically performed. For each patient, MRIs of the brain, optic nerve and spinal cord were performed at baseline, within 8 days of an NMOSD attack (if the patient suffered an NMOSD attack) and at the end of the RCP (month 6.5). MRIs were read centrally for new gadolinium-enhancing T1 (new Gad-T1) lesions by two independent, blinded-to-treatment neuroradiologists. Attacks were adjudicated by an expert committee.

Complete MRI data were available for 192 of 230 participants (83%), 42 of whom had an adjudicated attack (22 myelitis, 14 optic neuritis, 6 multidomain). Inter-rater agreement between the two neuroradiologists for gadolinium-enhancing lesions was 98% for brain, 95% for spinal cord, and 90% for optic nerve.

Of the 42 participants who had an adjudicated NMOSD attack, new Gad-T1 MRI lesions corresponding to the affected clinical domain were found in 19/22 (86%) of myelitis and 11/14 (79%) of optic neuritis attacks. During optic neuritis attacks, asymptomatic, new Gad-T1 lesions were simultaneously observed in 4/14 (29%) spinal cord and 1/14 (7%) brain MRIs. During myelitis attacks, asymptomatic, new Gad-T1 lesions were simultaneously observed in 6/22 (27%) optic nerve and 3/22 (14%) brain MRIs. Thus, asymptomatic Gad-T1 lesions were detected outside the symptomatic attack domain in about one-third of these participants.

Of the 150 participants without an adjudicated attack, new Gad-T1 MRI lesions were observed. New Gad-T1 MRI lesions were observed in the brain (3%), spinal cord (18%) and optic nerve (51%) of these participants at the end of randomized controlled period.

These data demonstrate that subclinical MRI lesions outside the symptomatic domain occurred during NMOSD attacks, and new MRI lesions were detected in some patients without NMOSD attacks. These data suggest that, contrary to the current understanding of NMOSD, subclinical radiographic disease activity in NMOSD is not uncommon. Analyses of full neuraxis MRI imaging in the clinical trial provides information on a potential role of NMI in NMOSD disease management and treatment.

### REFERENCES CITED IN THE DISCLOSURE

1. Cree B A, Bennett J L, Sheehan M, et al. Placebo-controlled study in neuromyelitis optica-ethical and design considerations. Mult Scler 2016; 22: 862-72.
2. Wingerchuk D M, Lennon V A, Lucchinetti C F, Pittock S J, Weinshenker B G. The spectrum of neuromyelitis optica. Lancet Neurol 2007; 6: 805-15.
3. Fujihara K, Misu T, Nakashima I, et al. Neuromyelitis optica should be classified as an astrocytopathic disease rather than a demyelinating disease. Clin Exp Neuroimmunol 2012; 3: 58-73.
4. Weinshenker B G, Wingerchuk D M, Vukusic S, et al. Neuromyelitis optica IgG predicts relapse after longitudinally extensive transverse myelitis. Ann Neurol 2006; 59: 566-9.
5. Bennett J L, Lam C, Kalluri S R, et al. Intrathecal pathogenic anti-aquaporin-4 antibodies in early neuromyelitis optica. Ann Neurol 2009; 66: 617-29.
6. Jarius S, Frederikson J, Waters P, et al. Frequency and prognostic impact of antibodies to aquaporin-4 in patients with optic neuritis. J Neurol Sci 2010; 298: 158-62.
7. Saadoun S, Waters P, Bell B A, Vincent A, Verkman A S, Papadopoulos M C. Intra-cerebral injection of neuromyelitis optica immunoglobulin G and human complement produces neuromyelitis optica lesions in mice. Brain 2010; 133: 349-61.
8. Bennett J L, O'Connor K C, Bar-Or A, et al. B lymphocytes in neuromyelitis optica. Neurol Neuroimmunol Neuroinflamm 2015; 2: e104.
9. Trebst C, Jarius S, Berthele A, et al. Update on the diagnosis and treatment of neuromyelitis optica: recommendations of the Neuromyelitis Optica Study Group (NEMOS). J Neurol 2014; 261: 1-16.
10. Damato V, Evoli A, Iorio R. Efficacy and Safety of Rituximab Therapy in Neuromyelitis Optica Spectrum Disorders: A Systematic Review and Meta-analysis. JAMA Neurol 2016; 73: 1342-8.
11. Ciron J, Audoin B, Bourre B, et al. Recommendations for the use of Rituximab in neuromyelitis optica spectrum disorders. Rev Neurol (Paris) 2018; 174: 255-64.
12. Cree B A, Lamb S, Morgan K, Chen A, Waubant E, Genain C. An open label study of the effects of rituximab in neuromyelitis optica. Neurology 2005; 64: 1270-2.
13. Palanichamy A, Jahn S, Nickles D, et al. Rituximab efficiently depletes increased CD20-expressing T cells in multiple sclerosis patients. J Immunol 2014; 193: 580-6.
14. Millen B, Dmitrienko A. Chain procedures: a class of flexible closed testing procedures with clinical trial applications. Stat Biopharm Res 2012; 3: 14-30.
15. Patra K, Cree B A C, Katz E, Pulkstenis E, Dmitrienko A, Cutter G. Statistical considerations for an adaptive design for a serious rare disease. Ther Innov Regul Sci 2016; 50: 375-84.
16. Schiopu E, Chatterjee S, Hsu V, et al. Safety and tolerability of an anti-CD19 monoclonal antibody, MEDI-551, in subjects with systemic sclerosis: a phase I, randomized, placebo-controlled, escalating single-dose study. Arthritis Res Ther 2016; 18: 131.
17. Wingerchuk D M, Lennon V A, Pittock S J, Lucchinetti C F, Weinshenker B G. Revised diagnostic criteria for neuromyelitis optica. Neurology 2006; 66: 1485-9.
18. Cree B A. Placebo controlled trials in neuromyelitis optica are needed and ethical. Mult Scler Relat Disord 2015; 4: 536-45.
19. Bretz F, Maurer W, Brannath W, Posch M. A graphical approach to sequentially rejective multiple test procedures. Stat Med 2009; 28: 586-604.
20. Genentech. Ocrevus prescribing information. 2017. https://www.gene.com/download/pdf/ocrevus_prescribing.pdf (accessed Feb. 28, 2019).
21. Hauser S L, Bar-Or A, Comi G, et al. Ocrelizumab versus interferon beta-la in relapsing multiple sclerosis. N Engl J Med 2017; 376: 221-34.
22. Montalban X, Hauser S L, Kappos L, et al. Ocrelizumab versus placebo in primary progressive multiple sclerosis. N Engl J Med 2017; 376: 209-20.
23. Genentech. Rituxan prescribing information. 1997. https://www.gene.com/download/pdf/rituxan_prescribing.pdf (accessed Feb. 28, 2019)
24. ClinicalTrials.gov. Efficacy and safety study as monotherapy of SA237 to treat NMO and NMOSD. 2014. https://clinicaltrials.gov/ct2/show/NCT02073279 (accessed Jan. 25, 2019).
25. ClinicalTrials.gov. Efficacy and safety study as add-on therapy of SA237 to treat NMO and NMOSD. 2014. https://clinicaltrials.gov/ct2/show/NCT02028884 (accessed Jan. 25, 2019).
26. ClinicalTrials.gov. A double blind trial to evaluate the safety and efficacy of eculizumab In relapsing NMO Patients (PREVENT study). 2013. https://clinicaltrials.gov/ct2/showNCT01892345 (accessed Jan. 25, 2019).
27. Paul F, Murphy O, Pardo S, Levy M. Investigational drugs in development to prevent neuromyelitis optica relapses. Expert Opin Investig Drugs 2018; 27: 265-71.

1. A method of treating neuromyelitis optica spectrum disorder (NMOSD), the method comprising:
administering VIB551 to a patient in need of treatment for NMOSD, wherein the VIB551 is administered intravenously at a dose of 300 mg every 6 months; and
treating the NMOSD.

2. The method of claim 1, wherein the treating is a reduction in worsening of Kurtzke Expanded Disability Severity Scale (EDSS) in the patient.

3. The method of claim 2, wherein the reduction in worsening of EDSS in the patient is:
a worsening of fewer than 2 points in EDSS score if the patient has a baseline score of 0;
a worsening of fewer than 1 point if the patient has a baseline score of 1 to 5; or
a worsening of less than 0.5 point if the patient has a baseline score of 5.5 or more.

4. The method of claim 1, wherein the treating is a reduction in number of active magnetic resonance imaging (MRI) lesions.

5. The method of claim 4 wherein the active MRI lesions are enlarging T2 MRI lesions.

6. The method of claim 1, wherein the treating is a reduction in number of new MRI lesions.

7. The method of claim 1, wherein the treating is a reduction in worsening of modified Rankin Score in the patient.

8. The method of claim 1, wherein the treating is a reduction in frequency of in-patient hospitalizations of the patient related to NMOSD.

9. The method of claim 1, wherein the treating is a reduction of risk of an NMOSD-related attack of the patient.

10. The method of claim 9, wherein the NMOSD-related attack is characterized by appearance of a new symptom or worsening of an existing symptom related to NMOSD.

11. The method of claim 10, wherein the symptom is an eye symptom.

12. The method of claim 11, wherein the eye symptom is eye pain, blurred vision, loss of vision, or appearance of an optic nerve lesion detected by MRI.

13. The method of claim 10, wherein the symptom is a spinal cord symptom.

14. The method of claim 13, wherein the spinal cord symptom is deep or radicular pain, extremity paraesthesia, weakness, sphincter dysfunction, Lhermitte's sign, or a spinal cord lesion detectable by MRI.

15. The method of claim 10, wherein the symptom is a brain or brain stem symptom.

16. The method of claim 15, wherein the brain or brainstem symptom is nausea, double vision, oculomotor palsy, vertigo, intractable vomiting, intractable hiccups, dysarthria, dysphagia, weakness, encephalopathy, hypothalamic dysfunction, or a brain or brain stem lesion detectable by MRI.

17. The method of claim 9, wherein the reduction of risk of the NMOSD-related attack is between 60 and 85%.

18. The method of claim 17, wherein the reduction of risk of the NMOSD-related attack is between 65 and 75%.

19. The method of claim 18, wherein the reduction of risk of the NMOSD-related attack is 70%.

20. The method of claim 17, wherein the reduction of risk of the NMOSD-related attack is 80%.

21. The method of claim 9, wherein the reduction of risk of the NMOSD-related attack is a probability of greater than 75% that the patient will have no attack within at least 6 months following the administering.

22. The method of claim 21, wherein the reduction of risk of the NMOSD-related attack is a probability of greater than 80% that the patient will have no attack within at least 6 months following the administering.

23. The method of claim 22, wherein the reduction of risk of the NMOSD-related attack is a probability of greater than 85% that the patient will have no attack within at least 6 months following the administering.

24. The method of claim 9, wherein the reduction of risk of the NMOSD-related attack is a reduction of annualized risk of NMOSD-related attack in the patient to between 0.18 and 0.07.

25. The method of claim 24, wherein the reduction of risk of the NMOSD-related attack is a reduction in annualized risk of NMOSD-related attack in the patient to between 0.15 and 0.08.

26. The method of claim 25, wherein the patient is AQP4-IgG seropositive and the reduction in annualized risk of the NMOSD-related attack is between 0.15 and 0.11.

27. The method of claim 24, wherein the patient is AQP4-IgG seronegative and the reduction in annualized risk of the NMOSD-related attack is between 0.07 and 0.09.

28. The method of claim 1, wherein the treating is a reduction in optic neuritis.

29. The method of claim 1, wherein the treating is a reduction of severity of NMOSD-related attacks.

30. The method of claim 29, wherein the reduction of severity of NMOSD-related attacks is the reduction in NMOSD-related attacks graded as major.

31. The method of claim 29, wherein the reduction of severity of NMOSD-related attacks is the reduction in NMOSD attacks requiring in-patient hospitalization.

32. The method of claim 1, wherein the treating is a decrease in NMOSD-related pain in the patient.

33. The method of claim 32, wherein the decrease in NMOSD-related pain is determined by measuring pain in legs of the patient.

34. The method of claim 1, wherein two weeks prior to the administering the 300 mg VIB551 every 6 months, an initial 300 mg VIB551 dose is administered to the subject

35. The method of claim 34, wherein oral corticosteroids are co-administered to the patient with the initial 300 mg VIB551 dose.

36. The method of claim 1, wherein the patient is AQP4-IgG seropositive

37. The method of claim 36, wherein the patient is screened for AQP4-IgG prior to the administering of VIB551.

38. A method of reducing active Mill lesions in a patient diagnosed with NMOSD, the method comprising:
administering VIB551 to a patient in need of treatment for NMOSD, wherein the VIB551 is administered intravenously at a dose of 300 mg every 6 months; and
reducing the MRI lesions in the patient.

39. The method of claim 38, wherein the active MRI lesions are enlarging T2 MRI lesions.

40. The method of claim 38, wherein the active MRI lesions comprise new Mill lesions.

41. The method of claim 38, wherein two weeks prior to the administering of the 300 mg VIB551 every 6 months, an initial 300 mg VIB551 dose is administered to the subject.

42. The method of claim 41, wherein oral corticosteroids are co-administered to the patient with the initial 300 mg VIB551 dose.

43. The method of claim 42, wherein the oral corticosteroids are administered daily for at least 2 weeks.

44. The method of claim 38, wherein the patient is AQP4-IgG seropositive.

45. The method of claim 38, wherein the reducing active Mill lesions in a patient is a reduction in the number of new MRI lesions in the patient.

46. A method of reducing AQP4-IgG titers in a AQP4-IgG⁺ patient in need of treatment for NMOSD, the method comprising:
administering VIB551 to a patient in need of treatment for NMOSD, wherein the VIB551 is administered intravenously at a dose of 300 mg every 6 months; and
reducing the AQP4-IgG titers in the patient.

47. A method of treating a patient diagnosed with NMOSD, the method comprising:
administering VIB551 to a patient in need of treatment for NMOSD, wherein the VIB551 is administered at a dose that: (i) depletes at least 90% of circulating CD20+ B cells for at least six months, and (ii) does not increase risk of infections in the patient; and
treating the NMOSD.

48. The method of claim 47, wherein the VIB551 further depletes peripheral blood CD20⁻ plasmablasts and plasma cells within 8 days following the administering.

49. The method of claim 47, wherein the dose is 300 mg.

50. The method of claim 48, wherein the dose is administered intravenously.

51. A method of reducing NMOSD-related disability in a patient diagnosed with NMOSD, the method comprising:
administering VIB551 to a patient in need of treatment for NMOSD, wherein the VIB551 is administered intravenously at a dose of 300 mg every 6 months; and
reducing the NMOSD-related disability in the patient.

52. The method of claim 51, wherein the reducing the NMOSD-related disability in the patient is a reduction in rate of worsening of NMOSD-related disability in the patient.

53. The method of claim 51, wherein the reducing the NMOSD-related disability in the patient is a lessening of NMOSD-related disability in the patient.

54. The method of any of claims 51 to 53, wherein the NMOSD-related disability is neurological disability.

55. The method of any of claims 51 to 53, wherein the reducing the NMOSD-related disability is determined using EDSS.

56. The method of claim 54, wherein the reducing the NMOSD-related disability is determined using modified Rankin Scale (mRS).

57. The method of any of claims 51 to 53, wherein the reducing the NMOSD-related disability is determined using mRS and EDSS.

58. The method of any of claims 51 to 53, wherein two weeks prior to the administering of the 300 mg VIB551 every 6 months, an initial 300 mg VIB551 dose is administered to the subject.

59. The method of claim 58, wherein oral corticosteroids are co-administered to the patient with the initial 300 mg VIB551 dose.

60. The method of claim 59, wherein the oral corticosteroids are administered daily for at least 2 weeks.

61. The method of claim 51, wherein the reducing the NMOSD-related disability in the patient is detectable within 6 to 12 months following the administering of a first dose of 300 mg of the VIB551.

62. The method of claim 61, wherein the reducing the NMOSD-related disability in the patient is detectable within 6 to 8 months following the administering of the first dose of 300 mg of the VIB551.

63. The method of claim 62, wherein the reducing the NMOSD-related disability in the patient is detectable within 6 to 7 months following the administering of the first dose of 300 mg of the VIB551.

64. The method of claim 1, wherein the treating is a reduction of NMOSD-related damage in the patient.

65. The method of claim 64, wherein the NMOSD-related damage is a clinically asymptomatic new MRI lesion.

66. The method of claim 65, wherein the clinically asymptomatic new Mill lesion occurs in the patient in the absence of symptoms of an NMOSD attack.

67. The method of claim 64, wherein the NMOSD-related damage is associated with an NMOSD-related attack, wherein the patient experiences symptoms of the NMOSD-related attack; and
wherein the NMOSD-related damage comprises a clinically asymptomatic new MRI lesion in a domain other than the domain in which the patient experiences symptoms of the NMOSD-related attack.

68. The method of claim 67, wherein the NMOSD-related damage further comprises a new MRI lesion in the domain in which the patient experiences symptoms of the NMOSD attack.

69. The method of claim 40, wherein at least one of the new MRI lesions is an asymptomatic MRI lesion.

70. A method of monitoring NMOSD progression in a patient diagnosed with NMOSD, the method comprising:
determining a first and a second number of MRI lesions in the patient; and
identifying the NMOSD in the patient as progressing if the second number of MRI lesions is greater than the first number of MRI lesions or identifying the NMOSD in the patient as non-progressing if the second number of MRI lesions is not greater than the first number of MRI lesions.

71. The method of claim 70, wherein the first and the second number of MRI lesions are determined at a time interval of between 6 and 24 months; and
wherein the patient is clinically asymptomatic throughout the time interval.

72. The method of claim 71, wherein the time interval is between 6 and 12 months.

73. The method of claim 72, wherein the time interval is approximately 6 months.

74. The method of claim 71, wherein the NMOSD in the patient is identified as progressing and wherein the patient is diagnosed as having had an NMOSD attack.

75. The method of claim 70 or 71, wherein the NMOSD is identified as progressing, and wherein the method further comprises a step of treating the patient.

76. The method of claim 75, wherein the treating the patient comprises administering VIB551 to the patient intravenously at a dose of 300 mg every 6 months.

77. The method of claim 70, wherein the first number of MRI lesions is determined prior to a first dose of a treatment.

78. The method of claim 77, wherein the first number of MRI lesions is determined prior to a first dose of a treatment and wherein the second number of MRI lesions is determined between 6 and 24 months following the first dose of the treatment.

79. The method of claim 78, wherein the patient is clinically asymptomatic from the first dose of the treatment to the between 6 and 24 months following the first dose of the treatment.

80. The method of claim 78 or 79, wherein the NMOSD is identified as non-progressing and the patient is identified as a responder to the treatment.

81. The method of claim 78 or 79, wherein the NMOSD is identified as progressing and the patient is identified as a non-responder to the treatment.

82. The method of claim 81, wherein the patient is diagnosed with having had an NMOSD attack.

83. A method of identifying a test agent as suitable for treating NMOSD in a patient diagnosed with NMOSD, the method comprising:
determining a first number of MRI lesions in the patient at most 1 month prior to treating with the test agent
determining a second number of MRI lesions in the patient between 3 and 24 months following treating with the test agent; and
identifying the test agent as suitable for treating NMOSD if the second number of MRI lesions is the same as or fewer than the first number of MRI lesions, or identifying the test agent as not suitable for treating NMOSD if the second number of MRI lesions is greater than the first number of MRI lesions.

84. The method of claim 83, wherein the first number of MRI lesions is determined in the patient at most 2 weeks prior to the treating with the test agent.

85. The method of claim 83 or 84 wherein the second number of MRI lesions is determined in the patient between 6 and 12 months following treatment with the test agent.

86. The method of claim 85, wherein the second number of MRI lesions is determined in the patient approximately 6 months following treatment with the test agent.

87. A method of reducing NMOSD-related attacks in a patient in need of treatment for NMOSD, the method comprising:
administering VIB551 to a patient in need of treatment for NMOSD, wherein the VIB551 is administered intravenously at a dose of 300 mg every 6 months; and
reducing the NMOSD-related attacks in the patient.

88. The method of claim 87, wherein two weeks prior to the administering the 300 mg VIB551 every 6 months, an initial 300 mg VIB551 dose is administered to the patient.

89. The method of claim 88, wherein oral corticosteroids are co-administered to the patient with the initial 300 mg VIB551 dose.

90. The method of claim 89, wherein the oral corticosteroids are administered daily for at least 2 weeks.

91. The method of claim 87, wherein the reducing the NMOSD-related attacks in the patient comprises a reduction in number of NMOSD-related attacks suffered by the patient in a first time period relative to a second time period,
wherein the first time period occurs following administration of a first VIB551 dose,
wherein the second time period occurs preceding administration of the first VIB551 dose, and
wherein the first and the second time period are of an equal length in time.

92. The method of claim 88, wherein the reducing the NMOSD-related attacks in the patient comprises a reduction in number of NMOSD-related attacks suffered by the patient in a first time period relative to a second time period,
wherein the first time period occurs following administration of the initial VIB551 dose,
wherein the second time period occurs preceding the initial VIB551 dose, and
wherein the first and the second time period are of an equal length in time.

93. The method of claim 91 or 92, wherein the first and the second time period are 6 months.

94. The method claim 91 or 92, wherein the first and the second time period are 12 months.

95. The method claim 91 or 92, wherein the first and the second time period are 18 months.

96. The method claim 91 or 92, wherein the first and the second time period are 24 months.

97. The method of claim 91 or 92, wherein the NMOSD-related attacks suffered by the patient in the first and the second time period comprise any one or more of an optic neuritis, a myelitis, or a brainstem attack.

98. The method of claim 97, wherein one or more of the NMOSD-related attacks suffered by the patient are asymptomatic.
